SEQUENCE LISTING (23 sequences)

SEQ ID NO: 1 - DNA, 14 bp, Artificial Sequence
  palindromic unit
gctcgagctcgagc

SEQ ID NO: 2 - DNA, 15 bp, Artificial Sequence
  palindromic unit
gctagatgcatctag

SEQ ID NO: 3 - DNA, 74 bp, Artificial Sequence
  template sequence
gttaatattattcgacgggcctgctcagctcgagcttgcatcgtgcagccgaagcttgcacgcgtgctattaat

SEQ ID NO: 4 - DNA, 14 bp, Artificial Sequence
  primer sequence
gcacgcgtgcaagc

SEQ ID NO: 5 - DNA, 14 bp, Artificial Sequence
  cDNA oligo
gcttgcacgcgtgc

SEQ ID NO: 6 - DNA, 34 bp, Artificial Sequence
  Cas9 clone forward primer
gcaaatgggtcgcggatccccaaagaagaagcgg

SEQ ID NO: 7 - DNA, 32 bp, Artificial Sequence
  Cas9 clone reverse primer
cgagtgcggccgcaagctttcacaccttcctc

SEQ ID NO: 8 - DNA, 128 bp, Artificial Sequence
  sgRNA
gttttttttaatacgactcactatagggcacgggcagcttgccgggttttagagctagaaatagcaagttaaaataaggctagtccgttatcaacttgaaaaagtggcaccgagtcggtgcttttttt

SEQ ID NO: 9 - DNA, 128 bp, Artificial Sequence
  cgRNA
gttttttttaatacgactcactatagggtaaccgtgcggtcgtacgttttagagctagaaatagcaagttaaaataaggctagtccgttatcaacttgaaaaagtggcaccgagtcggtgcttttttt

SEQ ID NO: 10 - DNA, 89 bp, Artificial Sequence
  RCA Template NC-23
gggcacgggcagcttgccggtggaagctagatgcatctagcaagcgccgccactgatttcaccgcttcaagctagatgcatctagcaat

SEQ ID NO: 11 - DNA, 89 bp, Artificial Sequence
  RCA template NC-12
gctaccgggcagcttgcatcaataagctagatgcatctagcaagcgccgccactgatttcaccgcttcaagctagatgcatctagcaat

SEQ ID NO: 12 - DNA, 89 bp, Artificial Sequence
  RCA Template NC-0
gagaaacgagtgcggtcacagctaagctagatgcatctagcaagcgccgccactgatttcaccgcttcaagctagatgcatctagcaat

SEQ ID NO: 13 - DNA, 10 bp, Artificial Sequence
  RCA Primer NC-F
gtggcggcgc

SEQ ID NO: 14 - DNA, 25 bp, Artificial Sequence
  Survey or assay promer T7EI-F
ggagttccgcgttacataacttacg

SEQ ID NO: 15 - DNA, 28 bp, Artificial Sequence
  Survey or Assay promer T7EI-R
aacctcgactaaacacatgtaaagcatg

SEQ ID NO: 16 - DNA, 62 bp, Artificial Sequence
  Wild Type Cas9/sgRNA targeted genomic locus in      U2OS.EGFP cells
gggtgggccagggcacgggcagcttgccggtggtgcagatgaacttcagctcgatgcggttc

SEQ ID NO: 17 - DNA, 43 bp, Artificial Sequence
  mutated clone 1 of 7 of Cas9/sgRNA targeted      gemonic locus in U2OS.EGFP cells
gggtgggccagggcacgggcagcttgccggctcgatgcggttc

SEQ ID NO: 18 - DNA, 54 bp, Artificial Sequence
  mutated clone 2 of 7 of Cas9/sgRNA targeted      gemonic locus in U2OS.EGFP cells
gggtgggccagggcacggccggtggtgcagatgaacttcagctcgatgcggttc

SEQ ID NO: 19 - DNA, 56 bp, Artificial Sequence
  mutated clone 3 of 7 of Cas9/sgRNA targeted      gemonic locus in U2OS.EGFP cells
gggtgggccagggcagcttgccggtggtgcagatgaacttcagctcgatgcggttc

SEQ ID NO: 20 - DNA, 57 bp, Artificial Sequence
  mutated clone 4 of 7 of Cas9/sgRNA targeted      gemonic locus in U2OS.EGFP cells
gggtgggccagggcacgggcgccggtggtgcagatgaacttcagctcgatgcggttc

SEQ ID NO: 21 - DNA, 60 bp, Artificial Sequence
  mutated clone 5 of 7 of Cas9/sgRNA targeted      gemonic locus in U2OS.EGFP cells
gggtgggccagggcacgggcagcttcggtggtgcagatgaacttcagctcgatgcggttc

SEQ ID NO: 22 - DNA, 65 bp, Artificial Sequence
  mutated clone 6 of 7 of Cas9/sgRNA targeted      gemonic locus in U2OS.EGFP cells
gggtgggccagggcacgggcagcttgctcacggtggtgcagatgaacttcagctcgatgcggttc

SEQ ID NO: 23 - DNA, 61 bp, Artificial Sequence
  mutated clone 7 of 7 of Cas9/sgRNA targeted      gemonic locus in U2OS.EGFP cells
gggtccagggcacgggcagcttgatccggtggtgcagatgaacttcagctcgatgcggttc

(19) United States
(12) Patent Application Publication
(10) Pub. No.: US 2019/0142744 A1
(43) Pub. Date: May 16, 2019

(54) NUCLEIC ACID NANOCAGES, COMPOSITIONS, AND USES THEREOF

(71) Applicant: North Carolina State University, Raleigh, NC (US)

(72) Inventors: Zhen Gu, Raleigh, NC (US); Wujin Sun, Raleigh, NC (US)

(21) Appl. No.: 15/751,688

(22) PCT Filed: Aug. 10, 2016
(86) PCT No.: PCT/US16/46353

§ 371 Date: Feb. 9, 2018

Related U.S. Application Data
(60) Provisional application No. 62/203,123, filed on Aug. 10, 2015.

(51) Int. Cl. A61K 9/00; A61K 47/55; A61K 31/704; A61K 47/69; B82Y 5/00; A61K 9/51

(52) U.S. Cl. CPC A61K 9/0092; A61K 47/551; A61K 9/51; A61K 47/6925; B82Y 5/00; A61K 31/704

(57) ABSTRACT

Described herein are nucleic acid based nanoparticles that can contain a cargo molecule. The nanoparticles described herein can be used to deliver a cargo molecule to a cell or area of interest.

### CROSS-REFERENCE TO RELATED APPLICATIONS

This application claims the benefit of and priority to co-pending U.S. Provisional Patent Application No. 62/203,123, filed on Aug. 10, 2015, entitled “NUCLEIC ACID NANOCAGES, COMPOSITIONS, AND USES THEREOF,” the contents of which is incorporated by reference herein in its entirety.

### STATEMENT REGARDING FEDERALLY SPONSORED RESEARCH OR DEVELOPMENT

This invention was made with government support under grant number TR001111 awarded by the National Institutes of Health. The government has certain rights to this invention.

### SEQUENCE LISTING

This application contains a sequence listing filed in electronic form as an ASCII.txt file entitled 221404-2040_ST25, created on Aug. 8, 2016. The content of the sequence listing is incorporated herein in its entirety.

### BACKGROUND

Delivery of cargo molecules, such as pharmaceutical compounds and other molecules to cells, is desirable for many applications ranging from treatment of diseases and disorders to industrial applications. However, many pharmaceuticals and other molecules cannot be delivered without a facilitator. For example, some molecules and/or compounds alone cannot be taken up by a cell or must otherwise be delivered in toxic amounts to have sufficient accumulation within a cell for any beneficial effect to be observed. As such, there exists a need for improved delivery compositions and systems for improved delivery of cargo molecules.

### SUMMARY

Provided herein are nanocages that can include a single stranded (ss) nucleic acid molecule having one or more palindromic units, wherein the ss nucleic acid molecule can be configured to self-assemble into the nucleic acid nanocage. The nanocages can further include a cargo molecule, wherein the cargo molecule can be coupled to and/or encapsulated by the DNA nanocage. The nanocages can further include a nanocapsule having a release molecule; and a stimuli responsive shell, wherein the stimuli responsive shell encapsulates the release molecule, and wherein the nanocapsule can be coupled to or encapsulated by the nanocage. The stimuli for the responsive shell can be a pH or change in pH. The nucleic acid nanocages can further include a targeting moiety, wherein the targeting moiety can be coupled to the nucleic acid nanocage. The cargo molecule can be selected from the group of: a nucleic acid; an amino acid; a peptide; a polypeptide; an antibody; a ribonucleoprotein; an aptamer; a ribozyme; a guide sequence for a ribozyme that is capable of inhibiting translation or transcription of essential tumor proteins and genes; a hormone; an immunomodulator; an antipyretic; an anxiolytic; an antipsychotic; an analgesic; an antispasmodic; an anti-inflammatory; an anti-histamine; an anti-infective; a chemotherapeutic; and any permissible combination thereof. The cargo molecule can be doxorubicin. The targeting moiety can be folic acid or analogue thereof. The ss nucleic acid molecule further can have a plurality of GC-pair sequences. The cargo molecule can be a Cas9:sgRNA riboonucleoprotein complex. The ss nucleic acid molecule is at least partially complementary to the sgRNA of the Cas9:sgRNA riboonucleoprotein complex. The targeting moiety can include a linker molecule operatively coupled to a targeting molecule, wherein the linker molecule can be coupled to the nucleic acid nanocage. The targeting moiety can include only a targeting molecule, wherein the targeting moiety can be coupled to the nucleic acid nanocage. The nanogages can have a surface modifier disposed around the nucleic acid nanocage. The surface modifier can be configured to generate an anionic, cationic, or neutral surface charge in one or more surface areas on the nanocage.

Also provided herein are compositions that can include an amount of any of the nanocages that can include a single stranded (ss) nucleic acid molecule having one or more palindromic units, wherein the ss nucleic acid molecule can be configured to self-assemble into the nucleic acid nanocage and provided elsewhere herein and a carrier. The carrier can be a pharmaceutically acceptable carrier.

Also provided herein are methods of genome editing that can include the step of incubating a cell for a period of time with an amount of any of the nanocages that can include a single stranded (ss) nucleic acid molecule having one or more palindromic units, wherein the ss nucleic acid molecule can be configured to self-assemble into the nucleic acid nanocage and provided elsewhere herein, composition thereof, or formulation thereof as provided herein.

Also provided herein are pharmaceutical formulations that can include an amount of of any of the nanocages that can include a single stranded (ss) nucleic acid molecule having one or more palindromic units, wherein the ss nucleic acid molecule can be configured to self-assemble into the nucleic acid nanocage and provided elsewhere herein and a pharmaceutically acceptable carrier. The amount of the nanocage can be an effective amount. The amount of the nanocage can be an amount effective to treat or prevent a disease, disorder, or symptom thereof in a subject. The disease can be cancer.

Also provided herein are methods of treating or preventing a disease, disorder, or symptom thereof in a subject in need thereof that can include the step of administering an effective amount of any of the nanocages that can include a single stranded (ss) nucleic acid molecule having one or more palindromic units, wherein the ss nucleic acid molecule can be configured to self-assemble into the nucleic acid nanocage and provided elsewhere herein or a pharmaceutical formulation thereof to the subject in need thereof. The disease, disorder, or symptom there of is cancer.

Also provided herein are nanocages that can have a single stranded (ss) nucleic acid molecule having one or more palindromic units, where the ss nucleic acid molecule can be configured to self-assemble into the nucleic acid nanocage. The nanocages can further include a cargo molecule, where the cargo molecule is coupled to and/or encapsulated by the DNA nanocage. The nanocages can further have a nanocapsule that can include a release molecule and a stimuli responsive shell, where the stimuli responsive shell encapsulates the release molecule, and where the nanocapsule is coupled to or encapsulated by the nanocage. The stimuli for the responsive shell can be a pH or change in pH. The release molecule can be a DNase. The nucleic acid nanocage can further include a targeting moiety, wherein the targeting moiety can be coupled to the nucleic acid nanocage. The cargo molecule can be selected from the group including: a nucleic acid; an amino acid; a peptide; a polypeptide; an antibody; a ribonucleoprotein; an aptamer; a ribozyme; a guide sequence for a ribozyme that is capable of inhibiting translation or transcription of essential tumor proteins and genes; a hormone; an immunomodulator; an antipyretic; an anxiolytic; an antipsychotic; an analgesic; an antispasmodic; an anti-inflammatory; an anti-histamine; an anti-infective; a chemotherapeutic; and any permissible combination thereof. The cargo molecule can be a chemotherapeutic. The chemotherapeutic can be doxorubicin. The targeting moiety can be folic acid or analogue thereof. The ss DNA molecule can further include a plurality of GC-pair sequences. The targeting moiety can include a linker molecule operatively coupled to a targeting molecule, wherein the linker molecule can be coupled to the nucleic acid nanocage. The targeting moiety consists of a targeting molecule and wherein the targeting moiety can be coupled to the nucleic acid nanocage.

Also provided herein are pharmaceutical formulations that can include any of the nanocages that can have a single stranded (ss) nucleic acid molecule having one or more palindromic units, where the ss nucleic acid molecule can be configured to self-assemble into the nucleic acid nanocage as provided elsewhere herein and a pharmaceutically acceptable carrier. The amount can be an effective amount to treat or prevent a cancer. The cancer can be a folate positive, PSA positive, beta-HCG positive, GalNAc positive, melanoma antigen gp75 positive, keratin 19 positive, a human cytokeratin positive cancer, or a combination thereof.

Also provided herein are methods of treating or preventing a disease, disorder, or symptom thereof that can include the step of administering an effective amount of any of the nanocages that can have a single stranded (ss) nucleic acid molecule having one or more palindromic units, where the ss nucleic acid molecule can be configured to self-assemble into the nucleic acid nanocage as provided elsewhere herein or a pharmaceutical formulation there of as provided herein. The subject in need thereof can have or be suspected of having a folate positive, PSA positive, beta-HCG positive, GalNAc positive, melanoma antigen gp75 positive, keratin 19 positive, and/or human cytokeratin positive cancer.

Also provided herein are methods that can include the step of contacting a cancer cell with any of the nanocages that can have a single stranded (ss) nucleic acid molecule having one or more palindromic units, where the ss nucleic acid molecule can be configured to self-assemble into the nucleic acid nanocage as provided elsewhere herein or a pharmaceutical formulation there of as provided herein. The cancer cell can be a folate positive, PSA positive, beta-HCG positive, GalNAc positive, melanoma antigen gp75 positive, keratin 19 positive, and/or human cytokeratin positive cancer cell.

Also provided herein are nanocages that can have a single stranded (ss) nucleic acid molecule that can include one or more palindromic units, where the ss nucleic acid molecule can be configured to self-assemble into the nucleic acid nanocage. The nanocages can further include a cargo molecule, where the cargo molecule that can be coupled to and/or encapsulated by the DNA nanocage. The cargo molecule can be selected from the group of: a nucleic acid; an amino acid; a peptide; a polypeptide; an antibody; a ribonucleoprotein; an aptamer; a ribozyme; a guide sequence for a ribozyme that is capable of inhibiting translation or transcription of essential tumor proteins and genes; a hormone; an immunomodulator; an antipyretic; an anxiolytic; an antipsychotic; an analgesic; an antispasmodic; an anti-inflammatory; an anti-histamine; an anti-infective; a chemotherapeutic; and any permissible combination thereof. The cargo molecule can be a ribonucleoprotein. The cargo molecule can be a Cas9:sgRNA complex. At least part of the ss nucleic acid can be at least partially complementary to the sgRNA. At least part of the ss nucleic acid can be complementary to 1 to 23 nucleotides of the sgRNA. At least part of the ss nucleic acid can be complementary to 12 nucleotides of the sgRNA. The complementary nucleotides can be consecutive within the sgRNA. The nanocages can further contain a surface modifier disposed around the nucleic acid nanocage. The surface modifier can be configured to generate an anionic, cationic, or neutral surface charge in one or more surface areas on the nanocage. The surface modifier can be polyethylenimine.

Also provided herein are formulations containing an amount of any of the nanocages that can have a single stranded (ss) nucleic acid molecule that can include one or more palindromic units, where the ss nucleic acid molecule can be configured to self-assemble into the nucleic acid nanocage provided elsewhere herein. The formulation can further include a pharmaceutically acceptable carrier. The amount of the nanocage can be an amount effective to modify a genome.

Also provided herein are methods of treating or preventing a disease, disorder, or symptom thereof in a subject in need thereof that can include the step of administering an effective amount of any of the nanocages that can have a single stranded (ss) nucleic acid molecule that can include one or more palindromic units, where the ss nucleic acid molecule can be configured to self-assemble into the nucleic acid nanocage provided elsewhere herein or a formulation thereof.

Also provided herein are methods that can include the step of contacting a cell with an amount of any of the nanocages that can have a single stranded (ss) nucleic acid molecule that can include one or more palindromic units, where the ss nucleic acid molecule can be configured to self-assemble into the nucleic acid nanocage provided elsewhere herein or a formulation thereof.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows one embodiment of a nucleic acid nanocage as described herein.

FIGS. 2A and 2B show main components of one embodiment of a cocoon-like self-degradable DNA nanocage (also referred to herein as a DNA nanoclew), containing a DOX/FA-NCl/NCa, acid-triggered DOX release (FIG. 2A), and a schematic illustration of efficient delivery of DOX by DOX/FA-NCl/NCa to nuclei for cancer therapy (FIG. 2B): (I) internalization in endosomes; (II) pH-triggered degradation of the NCI for DOX release; (III) accumulation of DOX in cell nuclei.

FIG. 3 shows one embodiment of synthesis of a DNA nanocage (e.g. NCI) by rolling circle amplification (I) with a cyclized ssDNA a template and a DNA oligo as primer, long chain single stranded DNA containing repeated sequence of the template was synthesized. (II) The synthesized RCA product can self-assemble into nanoclew-like structure by intramolecular hybridization.

FIG. 4 shows a table with sequences of oligos where the 5′ and 3′ ends of the ssDNA template were mainly composed of A and T to enhance flexibility for ligation. GC/CG pairs were included into the drug loading sites to increase DOX loading capacity. The palindromic sequence was incorporated to help assemble the ssDNA product into a clew (nanocage).

FIGS. 5A-5C demonstrate cyclization of the ssDNA template. FIG. 5A: Lane 1, DNA ladder; Lane d2, 5′ phosphorylated ssDNA template; Lane 3, cyclized ssDNA template; Lane 4, circular ssDNA template treated with Exo I (FIG. 5A), an 0.8% agarose gel analysis of the RCA product (FIG. 5B), and stability of NCI (FIG. 5C): Lane 1, non-treated NCI; Lane 2, NCI treated with DMEM containing 10% FBS for 24 h; Lane 3, NCI treated with DMEM containing 10% FBS for 48 h.

FIGS. 6A-6D demonstrate (FIG. 6A) hydrodynamic size of NCI as determined by dynamic light scattering (DLS). Inset: atomic force microscopy (AFM) image of NCI. The scale bar is 500 nm. (FIG. 6B) demonstrates the hydrodynamic size of NCa. Inset: transmission electron microscopy (TEM) image of NCa. The scale bar is 10 nm. (FIG. 6C) demonstrates the circular dichroism (CD) spectra of native DNase I and NCa. (FIG. 6D) demonstrates DNA-degrading activities of NCa and cNCa at pH 7.4 and 5.4. Bars represent mean±standard deviation (n=3).

FIG. 7 shows a graph demonstrating the fluorescence spectra of DOX solution (10 μM) with increasing concentrations of NCI (0.15-2.4 μg/mL).

FIG. 8 shows a graph demonstrating DOX entrapment efficiency and loading capacity by NCI. The DOX entrapment efficiency is the ratio of (added DOX−DOX washout out in supernatant)/added DOX. Drug loading capacity is the weight ratio of loaded DOX/(loaded DOX+NCI). Bars represent mean±SD (n=3).

FIG. 9 shows a table demonstrating sizes and zeta potentials of different particles.

FIGS. 10A-10F shows TEM images of DNase I (FIG. 10A), NCa (FIG. 10B), and NCa after incubation at pH 5.4 for 2 h (FIG. 10C). Scale bar is 10 nm. Hydrodynamic size of native DNase I (FIG. 10D), NCa (FIG. 10E) and NCa after incubation at pH 5.4 for 2 h (FIG. 10F).

FIGS. 11A-11C show CLSM images of NCl/NCa assembly. NCI was loaded with DOX (DOX/NCI) and NCa was conjugated with AF488-NHS for imaging (AF488-NCa). Scale bar is 50 μm.

FIGS. 12A-12D show theydrodynamic size of NCl/NCa complexes (FIG. 12A). Inset: TEM image of an NCI/Au-NCa complex. The scale bar is 100 nm. The arrows indicate Au-NCa adsorbed on the NCI surface. (FIG. 12B) shows DOX release from DOX/NCl/NCa and DOX/NCI/cNCa at pH 7.4 and 5.4. Bars represent mean±SD (n=3). (FIGS. 12C-12D) shows AFM images of NCl/NCa complexes after incubation at pH 7.4 and 5.4 for 2 h. The scale bar is 500 nm.

FIGS. 13A-13D shows confocal laser scanning microscopy images of MCF-7 cells after incubation with DOX/FA-NCl/NCa for different times. Late endosome and lysosomes were stained with LysoTracker green. Red, DOX; green, endolysosome; blue, Hoechst 33342; yellow, colocalization of red and green pixels; magenta, colocalization of red and blue pixels. The scale bar is 10 μm.

FIGS. 14A-14C demonstrate (FIG. 14A) relative uptake efficiency of DOX/FA-NCl/NCa by MCF-7 cells. **, P<0.01 compared with the control. Bars represent mean±SD (n=3). (FIG. 14B) In vitro cytotoxicities of DOX/NCI, DOX/NCl/NCa, and DOX/FA-NCl/NCa against MCF-7 cells for 24 h. *, P<0.05. Bars represent mean±SD (n=6). (FIG. 14C) In vitro cytotoxicities of the blank FA-NCI, NCa, and FA-NCl/NCa against MCF-7 cells for 24 h. Bars represent mean±SD (n=6).

FIGS. 15A-15Y shows CLSM images of MCF-7 cells after incubation with DOX/FA-NCl/NCa for different time. The late endosome and lysosomes are stained with LysoTracker (green), while the nuclei were stained with Hoechst. Scale bars are 10 μm.

FIGS. 16A-16L shows CLSM images of MCF-7 cells after incubation with DOX/FA-NCI/AF488-NCa for different time. The nuclei were stained with Hoechst. Scale bars are 10 μm.

FIG. 17 shows a graph demonstrating the in vitro cytotoxicity of DOX/NCI/cNCa and DOX/NCl/NCa against MCF-7 cells for 24 h. *P<0.05. Bars represent mean±SD (n=6).

FIGS. 18A-18B shows one embodiment of a DNA NC mediated CRISPR-Cas9 delivery system. (FIG. 18A) shows preparation of Cas9/sgRNA/NC/PEI. I: The NC can be synthesized by RCA and loaded with the Cas9/sgRNA complex through Watson-Crick base pairing; II: PEI can be coated onto Cas9/sgRNA/NC for enhanced endosome escape, (FIG. 18) B demonstrates one embodiment of delivery of Cas9/sgRNA by the DNA NC based carrier to the nucleus of the cell for genome editing. I: Bind to cell membrane; II: Endocytosis; III: Endosome escape; IV; Transport into the nucleus; V: Search for target DNA locus in the chromosome and introduce double strand breaks for genome editing.

FIG. 19 shows an image of a SDS-PAGE (12%) gel containing purified Cas9. The purified Cas9 showed molecular weight of ˜160 KDa.

FIGS. 20A and 20B are gel images demonstrating Agarose gel electrophoresis (0.8%) of purified sgRNA (lane 1) and cgRNA (lane 2) (FIG. 20A) and Cas9 activity assay using linearized plasmid pCAG-EGFP (5556 bp) as substrate (FIG. 20B). It was observed that only Cas9 complexed with sgRNA can digest the plasmid DNA.

FIG. 21 shows a table containing sequences of DNA and oligos used in Example 2.

FIGS. 22A-22D demonstrate particle characterization of Cas9/sgRNA/NC-12/PEI. (FIG. 22A) demonstrates monitoring zeta potential of the Cas9/sgRNA/NC-12/PEI assembly process. Bars represent mean±SD (n=3). (FIG. 22B) demonstrates hydrodynamic size distribution of Cas9/sgRNA/NC-12/PEI. (FIG. 22C) shows an AFM image and (FIG. 22D) shows a TEM image of Cas9/sgRNA/NC-12/PEI with scale bars of 400 nm and 100 nm, respectively.

FIG. 23 shows a graph demonstrating optimization of PEI concentration for coating Cas9/sgRNA/NC-12 by measuring the zeta potential.

FIGS. 24A-24F demonstrates hydrodynamic size distributions (FIGS. 24D-24F) and AFM images (FIGS. 24A-24C) of NC-12, Cas9/sgRNA/NC-12 and Cas9/sgRNA/NC-12/PEI. Scale bar 400 nm.

FIGS. 25A-25D shows CLSM images demonstrating Cas9/sgRNA/NC-12/PEI assembly. Red for Cas9 stained with AF647, blue for NC-12 stained with Hoechst 33342 and green for PEI labeled with FITC. Scale bar is 20 μm.

FIGS. 26A-26F demonstrate CLSM images (FIGS. 26A-26D) of U2OS.EGFP cells incubated with Cas9/sgRNA/NC-12/PEI for 1 h, 2 h, 4 h and 6 h (Cas9 and sgRNA concentrations at 100 nM). Green for EGFP, red for Cas9 stained with AF647 and blue for nuclei stained with Hoechst 33342. Scale bar is 10 μm (FIG. 26A); relative Cas9/sgRNA/NC-12/PEI uptake by U2OS.EGFP cells in the presence of different endocytosis inhibitors (Cas9 and sgRNA concentrations at 100 nM) (FIG. 26E) (**P<0.01 as compared to the control group. Bars represent mean±SD (n=3)); and In vitro cell viability of U2OS.EGFP cells treated with Cas9/sgRNA/NC-12/PEI and Cas9/sgRNA/PEI by flow cytometry (FIG. 26F). The cells were stained with TO-PRO-3 live/dead stain after the treatment and analyzed by flow cytometry. Bars represent mean±SD (n=3).

FIGS. 27A-27P shows confocal laser scanning microscopy images of U2OS.EGFP cells incubated with Cas9/sgRNA/NC-12/PEI for 1 h, 2 h, 4 h and 6 h (Cas9 and sgRNA concentrations at 100 nM).

FIGS. 28A-28H demonstrate Genome editing by Cas9/sgRNA delivered by DNA NC (8 μg/mL) coated with PEI (10 μg/mL). (FIGS. 28A-28C) shows fluorescent microscope images and flow cytometry analysis (FIGS. 28D-28F) of U2OS.EGFP cells treated with Cas9/sgRNA/PEI and Cas9/sgRNA/NC-12/PEI (Cas9 and sgRNA concentrations at 100 nM). Green represents EGFP and blue represents nuclei stained with Hoechst 33342. Scale bar is 100 μm. (FIG. 28G) demonstrates the results of a T7EI assay of U2OS.EGFP cells treated with Cas9/gRNA/NC-12/PEI and Cas9/gRNA/PEI. (FIG. 28H) demonstrates the results of a EGFP disruption assay of Cas9/gRNA delivered by different DNA NCs. Percentages of EGFP negative cells after treating with Cas9/sgRNA/NC-23/PEI, Cas9/sgRNA/NC-12/PEI, Cas9/sgRNA/NC-0/PEI and Cas9/sgRNA/PEI at different Cas9/sgRNA molar ratios were profiled. Bars represent mean±SD (n=3).

FIG. 29 shows a graph demonstrating results of a flow cytometry analysis of U2OS.EGFP cells treated with formulations containing cgRNA, which did not show any EGFP disruption efficacy.

FIGS. 30A and 30B shows gel electrophoretic images demonstrating agarose gel electrophoresis (0.8%) of synthesized NC-23, NC-12 and NC-0 in lane 1, 2 and 3, respectively (FIG. 30A) and analysis of NC stability after incubating with Cas9/sgRNA for 24 h. Lane 1, 3, 5 were for untreated NC-23, NC-12 and NC-0 and lane 2, 4, 6 showed Cas9/sgRNA treated NC-23, NC-12 and NC-0, respectively (FIG. 30B).

FIGS. 31A-31F show images demonstrating in vivo delivery of Cas9/sgRNA into U2OS.EGFP xenograft tumors in nude mice. Tumor sections were collected 10 days after intratumoral injection of Cas9/sgRNA/NC-12/PEI. The EGFP was stained by FITC conjugated GFP antibody and nuclei were stained with Hoechst 33342. Scale bar is 50 μm.

FIGS. 32A-32C show images of Tissue section of tumor treated with Cas9/cgRNA/NC-12/PEI. The EGFP was stained by FITC conjugated GFP antibody and nuclei were stained with Hoechst 33342. The Scale bar is 50 μm.

FIG. 33 demonstrates results from DNA sequencing of Cas9/sgRNA targeted gemonic locus in U2OS.EGFP cells (SEQ ID NOs.: 16-23). Target sequence complementary to the sgRNA is underlined and PAM sequence is shown in bold. Mutations were detected in 7 out of 20 sequenced clones. Number of insertion/deletion as compared to the wild type sequence is shown on the right.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### Definitions

As used herein, “about,” “approximately,” and the like, when used in connection with a numerical variable, generally refers to the value of the variable and to all values of the variable that are within the experimental error (e.g., within the 95% confidence interval for the mean) or within ±10% of the indicated value, whichever is greater.

As used herein, “active agent” or “active ingredient” refers to a component or components of a composition to which the whole or part of the effect of the composition is attributed.

As used herein, “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “amphiphilic”, as used herein, refers to a molecule combining hydrophilic and lipophilic (hydrophobic) properties.

As used herein, “antibody” refers to a protein produced by B cells that is used by the immune system to identify and neutralize foreign compounds, which are also known as antigens. Antibodies are glycoproteins belonging to the immunoglobulin superfamily.

Antibodies, recognize and bind to specific epitopes on an antigen.

As used herein, “anti-infective” refers to compounds or molecules that can either kill an infectious agent or inhibit it from spreading. Anti-infectives include, but are not limited to, antibiotics, antibacterials, antifungals, antivirals, and antiproatozoans.

As used herein, “aptamer” refers to single-stranded DNA or RNA molecules that can bind to pre-selected targets including proteins with high affinity and specificity. Their specificity and characteristics are not directly determined by their primary sequence, but instead by their tertiary structure.

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The terms “Cas9” and “Cas9 polypeptide” can be used interchangeably herein to refer to an enzyme (wild-type or recombinant) that can exhibit least endonuclease activity (e.g. cleaving the phosphodiester bond within a polynucleotide) guided by a CRISPR RNA (crRNA) bearing complementary sequence to a target polynucleotide. Cas9 polypeptides are known in the art, and include Cas9 polypeptides from any of a variety of biological sources, including, e.g., prokaryotic sources such as bacteria and archaea. Bacterial Cas9 includes, Actinobacteria (e.g., Actinomyces naeslundii) Cas9, Aquificae Cas9, Bacteroidetes Cas 9, Chlamydiae Cas9, Chloroflexi Cas9, Cyanobacteria Cas9, Elusimicrobia Cas9, Fibrobacteres Cas9, Firmicutes Cas9 (e.g., Streptococcus pyogenes Cas9, Streptococcus thermophilus Cas9, Listeria innocua Cas9, Streptococcus agalactiae Cas9, Streptococcus mutans Cas9, and Enterococcus faecium Cas9), Fusobacteria Cas9, Proteobacteria (e.g., Neisseria meningitides, Campylobacter jejuni and lari) Cas9, Spirochaetes (e.g., Treponema denticola) Cas9, and the like. Archaea Cas 9 includes Euryarchaeota Cas9 (e.g., Methanococcus maripaludis Cas9) and the like. A variety of Cas9 and related polypeptides are known, and are reviewed in, e.g., Makarova et al. (2011) Nature Reviews Microbiology 9:467-477, Makarova et al. (2011) Biology Direct 6:38, Haft et al. (2005) PLOS Computational Biology I:e60 and Chylinski et al. (2013) RNA Biology 10:726-737; K. Makarova et al., An updated evolutionary classification of CRISPR-Cas systems. (2015) Nat. Rev. Microbio. 13:722-736; and B. Zetsche et al. Cpf1 is a single RNA-guided endonuclease of a class 2 CRISPR-Cas system. (2015) Cell. 163(3):759-771. Other Cas9 polypeptides can be Francisella tularensis subsp. novicida Cas9, Pasteurella multocida Cas9, mycoplasma gallisepticum str. F Cas9, Nitratifractor salsuginis str DSM 16511 Cas9, Parvibaculum lavamentivorans Cas9, Roseburia intestinalis Cas9, Neisseria cinera Cas9, Gluconacetobacter diazotrophicus Cas9, Azospirillum B510 Cas9, Spaerochaeta globus str. Buddy cas9, Flavobacterium columnare Cas9, Fluviicola taffensis Cas9, Bacteroides coprophilus Cas9, mycoplasma mobile Cas9, lactobacillus farciminis Cas9, Streptococcus pasteurianus Cas9, Lactobacillus johnsonii Cas9, Staphylococcus pseudintermedius Cas9, filifactor alocis Cas9, Treponema denticola Cas9, Legionella pneumophila str. Paris Cas9, Sutterella wadsworthensis Cas9, and Corynebacter diptheriae Cas9. The term “Cas9” includes a Cas9 polypeptide of any Cas9 family, including any isoform of Cas9. Amino acid sequences of various Cas9 homologs, orthologs, and variants beyond those specifically stated or provided herein are known in the art and are publicly available, within the purview of those skill in the art, and thus within the spirit and scope of this disclosure.

As used herein, “chemotherapeutic agent” or “chemotherapeutic” refer to a therapeutic agent utilized to prevent or treat cancer.

As used herein, “composition” can refer to a combination of an active agent(s) and another compound or composition, inert (for example, a detectable agent or label) or active, such as an adjuvant, and can encompass and be used interchangeably with formulation.

As used herein, “concentrated” used in reference to an amount of a molecule, compound, or composition, including, but not limited to, a chemical compound, polynucleotide, peptide, polypeptide, protein, antibody, or fragments thereof, that indicates that the sample is distinguishable from its naturally occurring counterpart in that the concentration or number of molecules per volume is greater than that of its naturally occurring counterpart.

As used herein, “control” is an alternative subject or sample used in an experiment for comparison purposes and included to minimize or distinguish the effect of variables other than an independent variable. A “control” can be positive or negative.

As used herein, “conjugate” or “conjugated” refer to a covalent bond or a bond of similar strength and permanence of a covalent bond. “Conjugate” also includes irreversible bonds between two or more molecules or compounds.

As used herein with reference to the relationship between DNA, cDNA, cRNA, RNA, and protein/peptides, “corresponding to” refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “coupled” or “coupled to” refers to the attachment of one molecule to another via one or more additional molecules. “Coupled” includes both “conjugate” and “operatively linked.”

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “derivative” refers to any compound comprising the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include compounds in which carboxyl groups in the parent compound have been derivatized to form methyl and ethyl esters, or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. “Derivatives” also includes extensions of the replacement of the cyclopentane ring with saturated or unsaturated cyclohexane or other more complex, e.g., nitrogen-containing rings, and extensions of these rings with side various groups.

As used herein, “diabodies” are dimeric scFvs and can have shorter linkers than monomeric scFvs.

As used herein, “differentially expressed,” refers to the differential production of RNA, including, but not limited to, mRNA, tRNA, miRNA, siRNA, snRNA, and piRNA transcribed from a gene or regulatory region of a genome or the protein product encoded by a gene as compared to the level of production of RNA by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “diluted” used in reference to a an amount of a molecule, compound, or composition including but not limited to, a chemical compound, polynucleotide, peptide, polypeptide, protein, antibody, or fragments thereof, that indicates that the sample is distinguishable from its naturally occurring counterpart in that the concentration or number of molecules per volume is less than that of its naturally occurring counterpart.

As used herein, “DNA molecule” includes nucleic acids/polynucleotides that are made of DNA.

As used herein, “dose,” “unit dose,” or “dosage” refers to physically discrete units suitable for use in a subject, each unit containing a predetermined quantity of the nanoparticle composition or formulation calculated to produce the desired response or responses in association with its administration.

As used herein, “dsFv” refers to an Fv with an engineered intermolecular disulfide bond.

As used herein, “effective amount” refers to the amount of a targeted photoacoustic compound or derivative thereof or auxiliary agent described herein that will elicit the diagnostic, biological or medical response of a tissue, system, animal, or human that is being sought by the researcher, veterinarian, medical doctor or other clinician. “Effective amount” includes that amount of a nanocage, formulations and compositions thereof or derivative thereof or auxiliary agent described herein that, when administered, is sufficient to deliver a cargo molecule to a cell or other target, and/or result in genome editing of a target cell, prevent development of, alleviate to some extent, one or more of the symptoms of a cancer or other abnormality being treated or diagnosed. The effective amount will vary depending on the exact chemical structure of the nanocage or auxiliary agent, the location of the target cell, type of target cell, gene or other genome region being modified, the severity and/or type of the cancer or other disease, disorder, syndrome, or symptom thereof being treated, the route of administration, the time of administration, the rate of excretion, the drug combination, the judgment of the treating physician, the dosage form, and the age, weight, general health, sex and/or diet of the subject to be treated. “Effective amount” can refer to the minimal amount of a composition provided herein needed to elicit the desired effect and can result in delivery or administration of a lesser amount of an active agent included within the composition as compared to if the free active agent were used alone (e.g. not associated with a delivery vehicle, such as that provided herein).

As used herein, “encapsulation,” “encapsulate,” “encapsulated” and the like refer to the confinement, containment, or association of one individual molecule or compound within another individual molecule or compound. In some embodiments, “encapsulation,” “encapsulate,” “encapsulated” and the like can refer to the confinement, containment, or association of a nanoparticle within a shell or release molecule within a stimuli responsive shell.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, “Fab fragment” refers to as an antibody fragment essentially equivalent to that obtained by digestion of immunoglobulins with an enzyme (e.g. papin).

The Fab fragment can be enzymatically or recombinantly produced. The heavy chain segment of the Fab fragment is referred to as the Fd fragment.

As used herein, “Fv fragment” refers to an antibody fragment that contains one VH and one VL domain connected to one another via noncovalent interactions.

As used herein, “gene” refers to a hereditary unit corresponding to a sequence of DNA that occupies a specific location on a chromosome and that contains the genetic instruction for a characteristic(s) or trait(s) in an organism. The term “gene” encompasses specific nucleotide sequences of a genome that are transcribed into an RNA product and are not translated into a protein as well as those genomic sequences that are transcribed into an RNA product yet are translated into a protein.

As used herein, the terms “guide polynucleotide,” “guide sequence,” or “guide RNA” can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, can be about or more than about 50%, 60%, 75%, 80%, 85%, 90%, 95%, 97.5%, 99%, or more. In some embodiments, the degree of complementarily between a guide polynucleotide and its corresponding target sequence, when optimally aligned using a suitable alignment algorithm can be less than about 50%, such as 40%, 30%, 20% or less. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide can include a nucleotide sequence that is complementary to a target DNA sequence. This portion of the guide sequence can be referred to as the complementary region of the guide RNA. In some contexts, the two are distinguished from one another by calling one the complementary region or target region and the rest of the polynucleotide the guide sequence or tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015). A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence or by any of the delivery systems (e.g. nanocges, etc.) provided elsewhere herein. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

A complementary region of the gRNA can be configured to target any DNA region of interest. The complementary region of the gRNA and the gRNA can be designed using a suitable gRNA design tool. Suitable tools are known in the art and are available to the skilled artisan. Some such tools are discussed elsewhere herein. As such, the constructs described herein are enabled for any desired target DNA so long as it is CRISPR compatible according to the known requirements for CRISPR targeting. In some cases the design of the complementary region does not need to consider secondary structure and can be designed as described in F. Ran et al., Genome engineering using the CRISPR-CAs9 system. Nat. Protocol. 8(11): 2281-2308.

Homology-directed repair (HDR) refers to a mechanism in cells to repair double-stranded and single stranded DNA breaks. Homology-directed repair includes homologous recombination (HR) and single-strand annealing (SSA) (Lieber. (2010) Annu. Rev. Biochem. 79: 181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks.

Error-prone DNA repair refers to mechanisms that can produce mutations at double-strand break sites. The Non-Homologous-End-Joining (NHEJ) pathways are the most common repair mechanism to bring the broken ends together (Bleuyard et al., (2006) DNA Repair 5: 1-12). The structural integrity of chromosomes is typically preserved by the repair, but deletions, insertions, or other rearrangements are possible. The two ends of one double-strand break are the most prevalent substrates of NHEJ (Kirik et al., (2000) EMBO J. 19: 5562-5566), however if two different double-strand breaks occur, the free ends from different breaks can be ligated and result in chromosomal deletions (Siebert & Puchta, (2002) Plant Cell 14:1121-1131), or chromosomal translocations between different chromosomes (Pacher et al., (2007) Genetics 175: 21-29).

It will also be appreciated that CRISPR can also be used to activate specific genes through CRISPR/synergistic activation mediator procedures. These procedures can utilize a guide polynucleotide that incorporates 2 MS2 RNA aptamers at the tetraloop and the stem-loop of the guide RNA such as that described in, but not limited to (Nature 517, 583-588 (29 Jan. 2015).

As used herein “heterogeneous” refers to a population of molecules, including nanoparticles, proteins, and polypeptides, or a population of subunits of a molecule that contains at least 2 molecules or subunits that are different from one another.

As used herein “homogenous” refers to a population of molecules, including nanoparticles, proteins, and polypeptides, or a population of subunits of a molecule in which all the molecules or subunits are identical to one another.

The term “hydrophilic”, as used herein, refers to substances that have strongly polar groups that readily interact with water.

The term “hydrophobic”, as used herein, refers to substances that lack an affinity for water; tending to repel and not absorb water as well as not dissolve in or mix with water.

As used herein “immunomodulator,” refers to an agent, such as a therapeutic agent, which is capable of modulating or regulating one or more immune function or response.

As used herein “induces,” “inducing,” or “induced” refers to activating or stimulating a process or pathway within a cell.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

The term “lipophilic”, as used herein, refers to compounds comprising an affinity for lipids.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “nanocage” refers to a structure that can be formed of a nucleic acid, e.g. a single stranded nucleic acid, that can fold and interact (e.g. conjugate or associate) with itself or other molecules (such as other nucleic acids) to form a three dimensional structure that can be multilayered and can have the appearance of a ball of yarn in some embodiments, and depending on the association of the molecules that compose the nanocage, one or more voids.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules having DNA and RNA that may be single-stranded or, more typically, double-stranded or a mixture of single- and double-stranded regions. In addition, polynucleotide as used herein refers to triple-stranded regions having RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs having unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “nucleic acid sequence” and “oligonucleotide” also encompasses a nucleic acid and polynucleotide as defined above.

As used herein, “operatively linked,” “operatively link,” and the like refer to an association of two or more molecules or compounds that is not a covalent bond. Types of associations between two or more molecules or compounds that are “operatively linked” includes ionic associations, electrostatic interactions, hydrostatic interactions, van der Waals interactions, and the like. “Operatively linked” also includes any reversible bonds or associations between two or more molecules or compounds such that a compound or molecule is releasable. This type of interaction is also referred to herein as “releasable” or a “releasable link.” Operatively linked,” “operatively link” and the like also include the association of encapuslation between two or more molecules.

As used herein, “overexpressed” or “overexpression” refers to an increased expression level of an RNA or protein product encoded by a gene as compared to the level of expression of the RNA or protein product in a normal or control cell.

As used herein “palindromic units” refer to nucleic acid sequences that are palindromes. The palindromic units can be polynucleotide sequences within a larger polynucleotide molecule that can be separated by non-palindromic nucleotide sequences. Each of the individual palindromic polynucleotide sequence within the larger polynucleotide molecule can be considered an individual “palindromic unit”.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “preventative” refers to hindering or stopping a disease or condition before it occurs or while the disease or condition is still in the sub-clinical phase.

As used herein, “purified” is used in reference to a nucleic acid sequence, peptide, or polypeptide or other compound that has increased purity relative to the natural environment or the environment in which it was produced in.

As used herein, “separated” refers to the state of being physically divided from the original source or population such that the separated compound, agent, particle, chemical compound, or molecule can no longer be considered part of the original source or population.

As used herein, “single-chain Fvs” refer to recombinant antibody fragments contain only the variable light chain (VL) and variable heavy chaing (VH) covalently connected to one another by a polypeptide linker. Either the VL or HL can be at the N-terminal domain. The polypeptide linker can be of variable length and composition, so long as the two variable domains are bridged without significant steric interference. Linkers can include stretches of glycine and serine residues with some glutamate or lysine residues interspersed. scFvs can be monomeric or dimeric (and form a diabody).

As used herein, “small molecule”, as used herein, refers to a molecule, such as an organic or organometallic compound, with a molecular weight of less than 2,000 Daltons, less than 1,500 Daltons, less than 1,000 Daltons, less than 750 Daltons, or less than 500 Daltons. The small molecule can be a hydrophilic, hydrophobic, or amphiphilic compound.

As used herein, “specific binding partner” or “binding partner” is a compound or molecule to which a second compound or molecule binds with a higher affinity than all other molecules or compounds.

As used herein, “specifically binds” or “specific binding” refers to binding that occurs between such paired species such as enzyme/substrate, receptor/agonist or antagonist, antibody/antigen, lectin/carbohydrate, oligo DNA primers/DNA, enzyme or protein/DNA, and/or RNA molecule to other nucleic acid (DNA or RNA) or amino acid, which may be mediated by covalent or non-covalent interactions or a combination of covalent and non-covalent interactions. When the interaction of the two species produces a non-covalently bound complex, the binding that occurs is typically electrostatic, hydrogen-bonding, or the result of lipophilic interactions. Accordingly, “specific binding” occurs between a paired species where there is interaction between the two which produces a bound complex comprising the characteristics of an antibody/antigen, enzyme/substrate, DNA/DNA, DNA/RNA, DNA/protein, RNA/protein, RNA/amino acid, receptor/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “targeting moiety” can refer to a moiety or molecule that localizes to or away from a specific local, cell, and/or other molecule. As used herein, “targeting moiety” can refer to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. The targeting moiety or a sufficient plurality of targeting moieties may be used to direct the localization of a particle or an active entity. The active entity may be useful for therapeutic, prophylactic, or diagnostic purposes.

As used herein, “therapeutic” refers to treating or curing a disease or condition.

As used herein, “underexpressed” or “underexpression” refers to decreased expression level of an RNA or protein product encoded by a gene as compared to the level of expression of the RNA or protein product in a normal or control cell.

As used herein, “wild-type” is the typical form of an organism, variety, strain, gene, protein, or characteristic as it occurs in nature, as distinguished from mutant forms that may result from selective breeding or transformation with a transgene.

As used herein “zeta potential” refers without limitation to a potential gradient that arises across an interface. This term especially refers to the potential gradient that arises across the interface at the surface of a nanoparticle, also referred to as surface charge. Migration velocity of the particles depends on the amount of the surface charges and the applied field strength. Particles having a positive zeta potential migrate toward the negative electrode, and likewise particles having a negative zeta potential migrate toward the positive electrode. To determine the rate of migration, migrating particles are irradiated with a laser in the electric field. The movement of the particles is measured in a frequency shift in the reflected light compared to the incident light. The amount of frequency shift is dependent on the migration speed and is the so-called Doppler frequency shift (Doppler effect). From the Doppler frequency, the wavelength, the scattering angle and the rate of migration of a particle can be derived. The electrophoretic mobility is determined by the ratio of the moving speed and the electric field strength. The zeta potential is directly proportional to the electrophoretic mobility and is typically reported in mV. “Zeta potential”, as used herein, is defined wherein the zeta potential and particle size distribution were measured by dynamic light scattering (DLS) using a 90Plus Particle Size Analyzer by Brookhaven Instruments.

Discussion

Delivery of compounds and molecules to cells has applicability ranging from treatment of disease to improving techniques used in research. While many delivery platforms exist, they are not without limitations. As such there is an omnipresent need for the development of platforms for delivery of compounds and molecules to cells.

With that said, described herein are nucleic acid nanocages that can self-assemble and can include a cargo molecule. The nanocages described herein can be formulated as a composition, including pharmaceutical compositions. The nanocages and compositions thereof can be delivered to a cell and can be administered to a subject in need thereof.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Nucleic Acid Nanocages

Described herein are nucleic acid nanocages containing a single stranded (ss) nucleic acid molecule, where the ss nucleic acid molecule contains one or more palindromic units. The palandromic units can mediate self-assembly of the nucleic acid nanocage. The nanocage can contain one or more cargo molecules. The nanocage can contain one or more targeting moieties. The nanocage can contain one or more nanocapsules, where the nanocapsule can contain a stimuli responsive shell that can encapsulate a release molecule.

The nanocages described herein can deliver a cargo molecule to a cell. In some embodiments, the nanocages can release a cargo molecule into the environment surrounding the cell. In other embodiments, the nanocages can be internalized by the cell. In these embodiments, the nanocages can release a cargo molecule inside the cell.

Nanocages

i. ss Nucleic Acid Molecule

The nanocages described herein can be composed of a single stranded (ss) nucleic acid molecule. The ss nucleic acid molecule can be a DNA molecule, a RNA molecule, or a combination thereof. The length of the ss nucleic acid molecule can be greater than about 50 nucleotides. The length of ss nucleic acid molecule can be greater than about 75 nucleotides in length. In some embodiments, the length of the nucleic acid molecule can range from about 75 nucleotides to about 70,000 nucleotides or larger. The nanocages described herein can contain one or more palindromic units. The palandromic unit(s) can be located at various positions in the ss nucleic acid molecule. The length of the palindromic unit(s) can be greater than 3 nucleotides. The length of the palindromic unit can be greater than 3 nucleotides. In some embodiments, the length of the palindromic unit(s) can range from 3 nucleotides to 50 nucleotides. In some embodiments, the length of the palindromic unit can be 14 nucleotides. In some embodiments, the palindromic unit(s) can have a sequence identical to any one of SEQ ID NOs.: 1 or 2.

| | |
|---|---|
| SEQ ID NO 1: | GCTCGAGCTCGAGC |
| SEQ ID NO 2: | GCTAGATGCATCTAG |

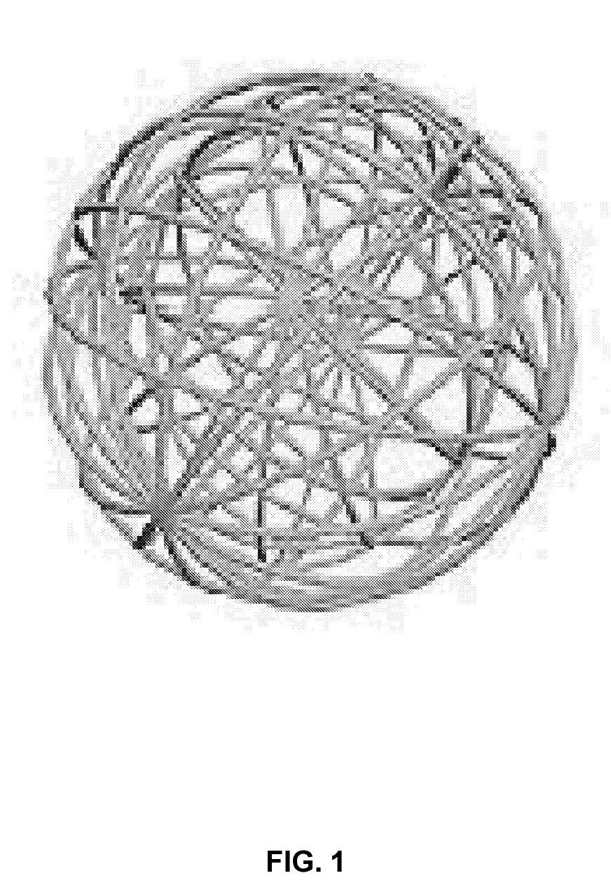

The nanocages described herein can self-assemble from a ss nucleic acid into a three dimensional nanocage. Self-assembly of the nanocages can be mediated by the palindromic unit(s) contained therein. The ss nucleic acid molecule can be configured such that when the nanocage self-assembles the ss nucleic acid molecule can be woven upon itself to form a substantially spherical structure with one or more voids of varying structure and size (see e.g. FIG. 1). In other embodiments, the nanocage can assume other three-dimensional shapes including, but not limited to a cube, cuboid, pyramid, prism, cylinder, and rhombohedron. In some embodiments, the nanocages described herein can have at least one dimension having a length of about 1 nm to about 1,000 nm. In some embodiments, the hydrodynamic size of the nanocage can range from about 1 nm to about 1000 nm. In other embodiments, the nanocages described herein can have at least one dimension having a length of greater than about 1,000 nm. In some of these embodiments, the at least one dimension of the nanocage can range from about 1,000 nm to about 1000 μm. Further, the hydrodynamic size of the nanocage can range from about 1,000 nm to about 1000 μm.

ii. Surface Modifiers

The nanocage can be coated with a surface modifier. A surface modifier can be applied to the nanocage to create particles having a charge or to create particles having different charges, i.e. to create two or more pluralities of particles wherein one plurality of particles may have a greater magnitude of charge (more negative or more positive) or may have an opposite charge as compared to a different plurality of particles Surface modifiers can be small molecule, oligomeric, or polymeric in nature. Surface modifiers can modify one or more properties related to charge, charge density, zeta potential, mechanical strength, rigidity, color, surface roughness, magnetic moment, or the presence and density of moieties on the surface. The moieties can include moieties that create specific or non-specific attractive (binding) interactions between particles. Exemplary moieties can include ligand and acceptor pairs such as antigen/antibody pairs, hydrogen bond donors and hydrogen bond acceptors, and cross-linking moieties. For example, a first plurality of particles can have a surface modifier presenting a plurality of acceptor moieties where a second plurality of particles can have a surface modifier presenting a plurality of targeting ligands that specifically bind the acceptor, thereby creating a strong attractive interaction between the different particles.

Likewise, nanocages having a surface presenting a plurality of hydrogen bond donors will have a strong attractive interaction to particles presenting a plurality of hydrogen bond acceptors.

In some embodiments the surface modifiers include one or more ligand/acceptor pairs, preferably binding with high affinity. High affinity ligand/acceptor pairs are known in the literature. Exemplary high affinity ligand/acceptor pairs include FK506/FKBP12, methotrexate/dihydro folate reductase, PPI-2458/methionine aminopeptidase, biotin/streptavidin tetramer, hirudin/thrombin, ZFV^{P}(0) F/carboxypeptidase, and chloroalkanes/haloalkane dehalogenases. Methods of determining high affinity binding pairs are known and can be used to identify high affinity ligand/acceptor pairs.

In some embodiments, the surface modifiers can include one or more hydrogen bond donors and/or one or more hydrogen bond acceptors. Exemplary hydrogen bond donors include moieties having available hydroxy or amino groups, including alcohols, phenols, carboxylic acids, primary and secondary amines, phosphonic acids, phosphoric acid esters, sulfonic acids, and sulfuric acids. Monosaccharides contain free —OH groups, therefore monosaccharides, disaccharides, oligosaccharides, and polysaccharides are exemplary hydrogen bond donors. Exemplary surface modifiers containing sugars include polymers such as alginate, chitosan, polyvinylalcohol, cellulose and cellulose derivatives such as methylcellulose, hydroxypropyl cellulose, hydroxyethyl cellulose, hydroxyethylmethyl cellulose, carboxymethyl cellulose, cellulose acetate, cellulose acetate phthalate, croscarmellose, hypromellose, and hydroxypropyl methyl cellulose), carrageenan, cyclodextrins, dextrin, polydextrose, other starches like corn starch, amylase, amylopectin, and sodium starch glycolate, sugars or small molecules like malic acid, trehalose, propylene glycol, glycerol, glycerol monostearate, sugars like sorbitol, ribose, arabinose, xylose, lyxose, allose, altrose, mannose, mannitol, glucose, dextrose, idose, galactose, talose, glucose, fructose, dextrose, dextrates, lactose, sucrose, and maltose, stearic acid, Vitamin E, and derivatives thereof. Exemplary hydrogen bond acceptors contain electronegative groups such as oxygen, nitrogen, sulfur, etc. with free valence electron pairs, including moieties containing for example —CO— and ═N—. Exemplary hydrogen bond acceptors include nitrogen containing groups such as amines, amides, imines, imides, nitriles and ureas as well as aromatic nitrogen-based functional groups such as pyridines, imidazoles, etc. as well as carboxylate groups (carboxylic acid, carboxylic ester), phosphonates, sulfoxides, sulfones, and carbamates.

In some embodiments, the nanocages can be coated with a biocompatible material having a positive charge or a negative charge. For example, to obtain oppositely-charged nanoparticles, polysaccharides such as chitosan and alginate, two polysaccharides that are used in humans, can be employed as surface modifiers to coat the nanoparticles to introduce both a positive and negative, respectively.

In some embodiments, different groups of nanocages can have opposite charges (opposite in sign) or can have charges that are of the same sign but having a different magnitude or amount of charge on the surface. In some embodiments, nanocages having an opposite charge, either by nature of the nanocage having a charge or via the charges from surface modifiers, can be combined to form a gel. The charged surface modifiers can include small molecules, oligomers, or polymers having a positive or a negative charge, preferably at or near a physiological pH. In some embodiments the charged surface modifier can be a surfactant on the surface of the particle. Exemplary positively charged surfactants (cationic surfactants) can include benzalkonium chloride (alkylbenzyldimethylammonium chloride); cetylpyridinium chloride; and cetyltrimethylammonium chloride (hexadecyltrimethylammoniurn chloride). Exemplary negatively charged surfactants (anionic surfactants) can include Dilauroylphosphoglycerol (1,2-Dilauroyl-sn-Glycero-3-[Phospho-rac-(l-glycerol)]; phosphatidic acid; saturated fatty acids, such as lauric acid, myristic acid, palmitic acid and stearic acid; unsaturated fatty acids, such as palmitoleic acid, oleic acid, linoleic acid and linolenic acid; deoxycholic acid; cholic acid; caprylic acid; glycocholic acid; glycodeoxycholic acid; lauroylsarcosine; and n-dodecyl sulfate.

In some embodiments, nanocages can be coated with surface modifiers containing one or more crosslinkers, for example, a thermally activated or UV activated crosslinker. Such crosslinkers include thermal crosslinkers which are activated at physiological temperatures or upon the application of heat. Such thermal crosslinkers can include multifunctional isocyanates, aziridines, multifunctional (meth)acrylates, and epoxy compounds. Exemplary crosslinkers include difunctional acrylates such as 1,6-hexanediol diacrylate or multifunctional acrylates such as are known to those of skill in the art. UV activated crosslinkers can also be used to crosslink the particles. Such UV crosslinkers can include benzophenones and 4-acry loxybenzophenones.

In some embodiments, the nanocages can be coated with surface modifiers that are charged polymers, i.e. cationic polymers or anionic polymers. Exemplary cationic polymers include linear and branched homopolymers and copolymers of polyallylamine (PAH); polyethyleneimine (PEI); poly(L-lysine) (PLL); a poly (L-arginine) (PLA); polyvinylamine; poly(vinylbenzyl-tri-Ci-C4-alkylammonium salt); poly(vinylpyridin), a poly(vinylpyridinium salt); a poly (,N-diallyl-N,N-di-Ci-C4-alkyl-ammoniumhalide); and/or polyaminoamide. Exemplary cationic polymers can include the copolymer of hydroxy ethyl cellulose and diallyldimethylammonium chloride, the copolymer of acrylamide and diallyldimethylammonium chloride, the copolymer of vinyl pyrrolidone and dimethylamino ethylmethacrylate methosulfate, the copolymer of acrylamide and betamethacrylyloxyethyl trimethyl ammonium chloride, the copolymer of polyvinyl pyrrolidone and imidazolimine methochloride, the copolymer of diallyldimethyl ammonium chloride and acrylic acid, the copolymer of vinyl pyrrolidone and methacrylamidopropyl trimethyl ammonium chloride, the methosulfate of the copolymer of methacryloyloxy ethyl trimethylammonium and methacryloyloxy ethyl dimethylacetylammonium, quaternized hydroxyethyl cellulose; dimethylsiloxane 3-(3-((3-cocoamidopropyl)dimethylammonio)-2-hydroxyprpoxy)propyl group terminated acetate; the copolymer of aminoethylaminopropylsiloxane and dimethyls iloxan; the polyethylene glycol derivative of aminoethylaminopropylsiloxane/dimethylsiloxan-copolymer and cationic silicone polymers. Exemplary anionic polymers include linear and branched homopolymers or copolymers of polyacrylic acid (PAA), polymethacrylic acid (PMA), maleic acid, fumaric acid, poly(styrenesulfonic acid) (PSS), polyamido acid, poly(2-acrylamido-2-methylpropanesulfonic acid) (poly-(AMPS)), alkylene polyphosphate, alkylene polyphosphonate, carbohydrate polyphosphate or carbohydrate polyphosphonate (e.g., teichoic acid).

Examples of synthetic anionic copolymers of methacrylic acid include a copolymerization product of an acrylic or methacrylic acid with a vinyl monomer including, for example, acrylamide, N,N-dimethyl acrylamide or N-vinylpyrrolidone. Exemplary anionic biopolymers or modified biopolymers include hyaluronic acid, glycosaminoglycanes such as heparin or chondroitin sulfate, fucoidan, poly-aspartic acid, poly-glutamic acid, carboxymethyl cellulose, carboxymethyl dextrans, alginates, pectins, gellan, carboxyalkyl chitins, carboxymethyl chitosans, and sulfated polysaccharides.

In some embodiments the nature of interaction and/or the density of surface modifiers on the particle can be adjusted to control the strength of the attractive interactions between the particles. In some embodiments these changes can be used to impact the physical properties of the resulting gel, i.e. the rigidity in non-shear or the fluidity in shear conditions, the overall mechanical strength of the gel, etc. Exemplary physical properties that can be modified include tensile strength, elongation, flexural strength, flexural modulus, viscosity under shear, etc.

In some embodiments, the surface modifier can be a stealth polymer. Stealth polymers, as used herein, refer to polymers that help prevent or reduce recognition of the conjugates by RES and/or Kupffer cells, for example by modification of the surface properties of conjugates to prevent opsonin interactions and subsequent phagocyte clearance. Stealth polymers can include natural, semi-synthetic, or synthetic polymers. Stealth polymers can include polysaccharides such as dextran, polysialic acid, hyaluronic acid, chitosan, heparin, and copolymers thereof. Stealth polymers can include polyvinyl pyrrolidone (PVP), polyvinyl alcohol (PVA), polyacrylamide (Pam), and copolymers thereof. Stealth polymers can include poly(alkylene glycols) and poly(alkylene oxides) such as poly(ethylene glycol), poly(propylene glycol), poly(ethylene oxide), or poly(propylene oxide). Stealth polymers can include copolymers of two or more stealth polymers.

In some embodiments, the surface modifier can be a biodegradable polymer. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water. Biodegradable polymers in the conjugate can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof.

Cargo Molecules

The nanocage can include a cargo molecule. Suitable cargo molecules include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. Other suitable cargo compounds include proteins and other molecules involved in genome editing, gene delivery, or gene regulation such as CAS9 and CAS9 complexes (e.g. CAS9:sgRNA ribonucleoprotien complexes), zinc finger nucleases, transcription activator-like effector nuclease and transcription factors.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbituates, hyxdroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylae, and sodium salicaylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, poluethyinterferon alfa-2b, maraviroc, raltegravir, dolutegravir, enfuvirtide, foscarnet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, aspargainase erwinia chyrsanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

The cargo molecule can be attached to, either directly or indirectly, the ss nucleic acid of the nanocage. In other embodiments, the cargo molecule can be intercalated into the DNA chains. In some embodiments, the cargo molecule can bind to the DNA via hybridization. In some embodiments, the cargo molecule can adsorb onto the DNA nanocage by electrostatic interaction. In some embodiments, the cargo molecule can be operatively coupled to a nucleic acid that is complementary to a portion of the ss nucleic acid of the nanocage. In some embodiments, the cargo molecule can be attached to the nanocage via a covalent interaction. In other embodiments, the cargo molecule can be attached to the nanocage via a non-covalent interaction.

Targeting Moieties

The nanocages described herein can include one or more targeting moieties conjugated to the nanocage. In some embodiments, the targeting moiety can be composed of only a targeting molecule that can be directly conjugated to the nanocage. In other embodiments, the targeting moiety can be composed of a targeting molecule that can be indirectly conjugated to the nanocage via a linker molecule that can be operatively coupled to the targeting molecule. The targeting moiety can be conjugated to the nanocage via non-covalent or covalent interactions between the targeting molecule and/or the linker molecule.

i. Targeting Molecules

The targeting molecule can be a protein such as an antibody or an antigen-binding fragment thereof. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The targeting molecule can have an affinity for a cell-surface receptor or cell-surface antigen on the target cells. The targeting molecule can be a nucleic acid targeting molecule. The targeting molecule can be a small molecule. The targeting molecule can result in internalization of the particle within the target cell.

The targeting molecule can specifically recognize and bind to a target molecule specific for a cell type, a tissue type, or an organ. The target molecule can be a cell surface polypeptide, lipid, or glycolipid. The target molecule can be a receptor that is selectively expressed on a specific cell surface, a tissue or an organ. Cell specific markers can be for specific types of cells including, but not limited to stem cells, skin cells, blood cells, immune cells, muscle cells, nerve cells, cancer cells, virally infected cells, and organ specific cells. The cell markers can be specific for endothelial, ectodermal, or mesenchymal cells. Representative cell specific markers include, but are not limited to cancer specific markers.

The targeting molecule can specifically bind to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.).

A tumor antigen may include a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Targeting molecules can include derivatives of known targeting molecules, for example derivatives having reactive coupling groups that can be used to bond the targeting molecule to the nanocage or to a polymer or other molecule. In some embodiments the targeting molecule targets the folate receptor, for example the targeting moiety can be folic acid or a folic acid derivative.

ii. Linker Molecules

In some embodiments, the targeting moiety can include a linker molecule to facilitate binding of a targeting molecule to the nanocage. The linker molecule can include homopolymers and copolymers, nucleic acids, proteins, and the like. In some embodiments, the linker can be a stable linker. In other embodiments, the linker can be a liable linker that can be degraded by changes in temperature, pH, or by enzymatic degradations.

Nanocapsules

The nanocage can include one or more nanocapsules that can contain a release molecule. The release molecule can facilitate the release of the cargo molecule from the nanocage. The nanocapsule can include a core that contains the release molecule and a stimuli responsive coating surrounding the core to form a stimuli responsive shell. The stimuli responsive shell can be directly responsive to one or more external stimuli. Stimulation of the stimuli responsive shell can result in degradation of the stimuli responsive shell, which can result in the release of the release molecule from the nanocapsule. The release molecule can then interact with the nanocage and/or other components of the nanocage to mediate and/or potentiate the release of the cargo molecule from the nanocage. External stimuli can include, pH, temperature, concentration of one or more chemical or enzymatic agents, radiation, UV light, light, etc.

i. Stimuli Responsive Shell

The stimuli responsive shell can be composed of a polymer matrix. The polymer matrix can be biocompatible and/or biodegradable. Representative polymers that can be contained in the stimuli responsive shell include, without limitation, homopolymers and copolymers of polysaccharides such as alginate, chitosan, dextran, mannan, pullulan, hyaluronic acid (HA), and xanthan gum; biodegradable polyesters such as polylactic acid, polyglycolic acid, poly(3-hydroxybutyrate), and polycaprolactone; acrylate and methacrylate polymers such as 2-(hydroxyethyl) methacrylate, and copolymers thereof. The rate of biodegradation can be adjusted by altering the ratio of repeat units in a copolymer. For example, when the polymeric matrix is poly(lactic-co-glycolic acid) (PLGA), the rate of biodegradation can be controllably varied from a few days to several months by varying the ratio of lactic acid and glycolic acid in the polymer. The polymers forming the polymeric matrix can in some embodiments be modified to provide or to enhance responsiveness to the one or more external stimuli. These are preferably hydrophilic polymers. For example, in some embodiments a biodegradable polymer such as a polysaccharide is modified to be pH responsive.

The rate of biodegradation of the stimuli responsive shell can be responsive to local pH. Examples of pH-sensitive polymers useful in drug delivery include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

The polymers forming the stimuli responsive shell can be further modified to provide or to increase responsiveness to the local pH. For example, in some embodiments the polymer is a modified polyhydroxylated polymer that is a polyhydroxylated polymer having reversibly modified hydroxyl groups, wherein the hydroxyl groups are modified to feature an acid degradable functional group. Exemplary acid degradable functional groups can be acetals, aromatic acetals, ketals, vinyl ethers, aldehydes, or ketones. The hydroxyl groups in the polyhydroxylated polymers are modified, thereby rendering the modified polyhydroxylated polymer acid degradable, pH sensitive and typically insoluble in water. The polyhydroxylated polymers can be preformed natural polymers or hydroxyl-containing polymers including, but not limited to, multiply-hydroxylated polymers, polysaccharides, carbohydrates, polyols, polyvinyl alcohol, poly amino acids such as polyserine, and other polymers such as 2-(hydroxyethyl)methacrylate. Exemplary polysaccharides that can be used to form modified polyhydroxylated polymers include, but are not limited to, dextran, mannan, pullulan, maltodextrin, starches, cellulose and cellulose derivatives, gums (e.g., xanthan, locust bean, etc.), and pectin. In one embodiment, the polysaccharides are dextran or mannan.

The reversible modification of the hydroxyl groups in modified polyhydroxylated polymers can be carried out to provide modified hydroxyl groups, wherein at least 20%, 25%, 30%, 40%, 50%, 60%, 70%, 75%, 80%, 85%, 90%, 95%, 98%, 99% or 100% of the hydroxyl groups in the polymer are modified.

The choice of the polyhydroxylated polymer and the degree of modification can be based upon such factors as ease of synthesis, solubility, commercially available reagents, the type of acid-degradable polymer desired, the loading efficiency, and/or dispersion of drug delivery systems comprised of the polymers, toxicity and the hydrolysis rates of the acetal linkage. It is preferred that the degradation products are biocompatible and biodegradable. For example, the degradation products should be non-immunogenic and non-toxic, for example, with the size and/or toxicity levels preferred by one having skill in the art for approved in vivo use.

The modified polyhydroxylated polymers can be modified polysaccharides with pendant acetals, thus providing acetal-derivatized polysaccharides. In some embodiments, the modified polyhydroxylated polymers are acetal-derivatized dextran, acetal-derivatized mannan or acetal-derivatized polyvinyl alcohols, preferably acetal-derivatized dextran.

The modified polymers having a modified functional (e.g., acetal or ketal) linkage at the modified hydroxyl groups should degrade by acid catalyzed hydrolysis into lower molecular weight compounds that can be completely excretable. The rate of hydrolysis of these polymers can be changed by varying the functional group (e.g., acetal or ketal) linkage from slow degrading to fast degrading, the degree of modification, or the hydrophobicity of the modification, thus providing a wide range of release kinetics for drug delivery. Thus, it is contemplated that a variety of acid degradable linkages with different acid-sensitivities can be incorporated onto the polymer backbones, allowing for greater control of the rate of polymer hydrolysis.

In some embodiments, the present acid degradable polymers described herein can have a significantly lower rate of degradation in solution at pH 7.4 than at pH 5. In some embodiment the polymers can have a degradation half-life at pH 5.0 of 5 minutes to 24 hours at 37° C., but a longer half-life at pH 7.4 of at least 12 hours to 250 days. In some embodiments, it can be useful for the polymers to have a half-life at pH 5.0, 37° C. of about 5-30 minutes, of about 2-5 hours, or of about 24 hours, while a half-life at pH 7.4, 37° C. of about 90 days, about 180 days, or about 250 days, in order to facilitate the rapid release of bioactive materials at acidic pH and slow release of bioactive materials at physiological pH. In some embodiments, the modified polyhydroxylated polymers are largely stable at pH higher than 7.4 but hydrolyze at a pH preferably about 5. In one embodiment, the modified polymers are soluble in common organic solvents to facilitate processing into a variety of materials. In another embodiment, these modified polymers are not water soluble.

Other matrix materials can be used, where degradation of the formulation is triggered by other stimuli, such as enzyme activity, redox conditions or photo irradiation.

ii. Release Molecules.

The nanocapsules can include a release molecule that can facilitate the degradation of the nanocage and/or other components contained in the nanocate to allow a cargo molecule to be released. Suitable release molecules include, without limitation, nucleases, proteases, lipases, amylases, oxidoreductases, and transferases. In some embodiments the release molecule can be a DNAse, such as DNAse I.

Nucleic Acid Nanocages Compositions

The nanocages described herein can be provided to a subject alone or contacted with a cell (in vivo or in vitro) or as an ingredient, such as an active ingredient, in a pharmaceutical formulation or other composition. As such, also described herein are pharmaceutical formulations containing one or more of the nanocages described herein. In some embodiments, the pharmaceutical formulations contain an effective amount of nanocages described herein. The pharmaceutical formulations can be administered to a subject in need thereof. In some embodiments, the subject in need thereof can have a cancer. In other embodiments the subject in need thereof can have a folate positive, PSA positive, beta-HCG positive, GalNAc positive, melanoma antigen gp75 positive, keratin 19 positive, and/or human cytokeratin positive cancer.

The pharmaceutical formulation can contain a homogenous population of nanocages. In these embodiments, all of the nanocages contained in the pharmaceutical formulation are the same. In other embodiments, the pharmaceutical formulation can contain a heterogeneous population of nanocages. In these embodiments, the population of nanocages contains at least two nanocages that are different from one another. The two different nanocages can vary from one another in the signaling molecule, the targeting moiety, and/or the linker contained therein.

Pharmaceutically Acceptable Carriers and Auxiliary Ingredients and Agents

The pharmaceutical formulations containing an effective amount of the nanocages described herein can further include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxyl methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to the effective amount of the nanocages, the pharmaceutical formulation can also include an effective amount of auxiliary active agents, including but not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. Suitable compounds for the auxiliary active agents have been previously described herein in relation to the nanocages.

Effective Amounts of the Nanocages and Auxiliary Agents

The pharmaceutical formulations can contain an effective amount of the nanocages and/or an effective amount of an auxiliary agent. In some embodiments, the effective amount ranges from about 0.001 pg of the nanocages to about 1,000 μg of nanocages. In other embodiments, the concentration of the nanocages effective amount ranges from about 1 nM to about 50 nM.

In embodiments where there is an auxiliary active agent contained in the pharmaceutical formulation in addition to the nanocages, the effective amount of the auxiliary active agent will vary depending on the auxiliary active agent. In some embodiments, the effective amount of the auxiliary active agent ranges from 0.001 micrograms to about 1 miligram. In other embodiments, the effective amount of the auxiliary active agent ranges from about 0.01 IU to about 1000 IU. In further embodiments, the effective amount of the auxiliary active agent ranges from 0.001 mL to about 1 mL. In yet other embodiments, the effective amount of the auxiliary active agent ranges from about 1% w/w to about 50% w/w of the total pharmaceutical formulation. In additional embodiments, the effective amount of the auxiliary active agent ranges from about 1% v/v to about 50% v/v of the total pharmaceutical formulation. In still other embodiments, the effective amount of the auxiliary active agent ranges from about 1% w/v to about 50% w/v of the total pharmaceutical formulation.

The auxiliary active agent can be included in the pharmaceutical formulation or can exist as a stand-alone compound or pharmaceutical formulation that is administered contemporaneously or sequentially with the nucleic acid nanocage, derivative thereof or pharmaceutical formulation thereof provided herein. In embodiments where the auxiliary active agent is a stand-alone compound or pharmaceutical formulation, the effective amount of the auxiliary active agent can vary depending on the auxiliary active agent used. In some of these embodiments, the effective amount of the auxiliary active agent ranges from 0.001 micrograms to about 1000 grams. In other embodiments, the effective amount of the auxiliary active agent ranges from about 0.01 IU to about 1000 IU. In further embodiments, the effective amount of the auxiliary active agent ranges from 0.001 mL to about 1 mL. In yet other embodiments, the effective amount of the auxiliary active agent ranges from about 1% w/w to about 50% w/w of the total auxiliary active agent pharmaceutical formulation. In additional embodiments, the effective amount of the auxiliary active agent ranges from about 1% v/v to about 50% v/v of the total pharmaceutical formulation. In still other embodiments, the effective amount of the auxiliary active agent ranges from about 1% w/v to about 50% w/v of the total auxiliary agent pharmaceutical formulation.

Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, and intradermal. Such formulations may be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. In some embodiments, this is a subject having cancer. In some embodiments, the cancer is folate positive cancer.

Where appropriate, the dosage forms described herein can be microencapsulated. The dosage form can also be prepared to prolong or sustain the release of any ingredient. In some embodiments, the nanocage can be the ingredient whose release is delayed. In other embodiments, the release of an auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water soluble polymer, water insoluble polymers, and/or pH dependent polymers, with or without water insoluble/water soluble non polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the nanocage, auxiliary active ingredient, and/or pharmaceutically acceptable salt thereof can be formulated with a parafinnic or water-misicible ointment base. In other embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the nanocages, derivative thereof, auxiliary active ingredient, and/or pharmaceutically acceptable salt thereof in a dosage form adapted for inhalation is in a particle-size-reduced form that is obtained or obtainable by micronization. In some embodiments, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient, which may be generated by various types of metered dose pressurized aerosols, nebulizers, or insufflators.

In some embodiments, the dosage forms are aerosol formulations suitable for administration by inhalation. In some of these embodiments, the aerosol formulation contains a solution or fine suspension of a nanocages, auxiliary active ingredient, and/or pharmaceutically acceptable salt thereof a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a nucleic acid nanocage, derivative thereof, auxiliary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once, once daily, or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the nanocage, auxiliary active ingredient, and/or pharmaceutically acceptable salt thereof, such a dosage form can contain a powder base such as lactose, glucose, trehalose, manitol, and/or starch. In some of these embodiments, the conjugate compound, derivative thereof, auxiliary active ingredient, and/or pharmaceutically acceptable salt thereof is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol formulations are arranged so that each metered dose of aerosol contains a predetermined amount of an active ingredient, such as the one or more of the compounds described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for injection (i.v., s.q., i.c.v., i.m. etc.), can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

For some embodiments, the dosage form contains a predetermined amount of a nucleic acid nanocage provided herein per unit dose. In an embodiment, the predetermined amount of the nucleic acid nanocage provided herein is an effective amount of the nanocages to diagnose, treat, prevent, or mitigate the symptoms of cancer. In other embodiments, the predetermined amount of the nanocages is an appropriate fraction of the effective amount of the active ingredient. Such unit doses may therefore be administered once or more than once a day. Such pharmaceutical formulations may be prepared by any of the methods well known in the art.

Methods of Using Nucleic Acid Nanocages and Compositions Thereof

The nanocages and pharmaceutical formulations thereof described herein can be used for the diagnosis, treatment, or prevention of a disease, disorder, syndrome, or a symptom thereof. In some embodiments, the nanocages or pharmaceutical formulations thereof can be used to diagnose, treat, or prevent a cancer or symptom thereof. The cancer can be a folate positive, PSA positive, beta-HCG positive, GalNAc positive, melanoma antigen gp75 positive, keratin 19 positive, and/or human cytokeratin positive cancer. The targeted nanocages and pharmaceutical formulations thereof can be used as a delivery conduit for treatment of a subject in need thereof and for delivery of genome editing molecules in a clinical and research setting.

An amount of the nanocages and pharmaceutical formulations thereof described herein can be administered to a subject in need thereof one or more times per day, week, month, or year. In some embodiments, the subject has one or more symptoms of a disease, condition, or syndrome. In some of these embodiments, the disease, condition, or syndrome can be cancer. In some embodiments, the cancer can be a folate positive, PSA positive, beta-HCG positive, GalNAc positive, melanoma antigen gp75 positive, keratin 19 positive, and/or human cytokeratin positive cancer. In some embodiments, the amount administered can be the effective amount of the nanocages or pharmaceutical formulations thereof. For example, the nanocages or pharmaceutical formulations thereof, can be administered in a daily dose. This amount may be given in a single dose per day. In other embodiments, the daily dose may be administered over multiple doses per day, in which each containing a fraction of the total daily dose to be administered (sub-doses). In some embodiments, the amount of doses delivered per day is 2, 3, 4, 5, or 6. In further embodiments, the compounds, formulations, or salts thereof are administered one or more times per week, such as 1, 2, 3, 4, 5, or 6 times per week. In other embodiments, the nanocages or pharmaceutical formulations thereof are administered one or more times per month, such as 1 to 5 times per month. In still further embodiments, the nanocages or pharmaceutical formulations thereof are administered one or more times per year, such as 1 to 11 times per year.

In embodiments where more than one of the nanocages, pharmaceutical formulations thereof, and/or auxiliary agent(s) are administered sequentially; the sequential administration may be close in time or remote in time. For example, administration of the second nanocages, pharmaceutical formulations thereof, and/or auxiliary agent(s) can occur within seconds or minutes (up to about 1 hour) after administration of the first agent (close in time). In other embodiments, administration of the second nanocages, pharmaceutical formulations thereof, and/or auxiliary agent(s) occurs at some other time that is more than an hour after administration of the first the nanocages or pharmaceutical formulations thereof.

The amount of the nanocages, pharmaceutical formulations thereof, and/or auxiliary agent(s) described herein can be administered in an amount ranging from about 0.01 mg to about 1 mg per day, as calculated as the free or unsalted pharmaceutical formulations. The amount of nanocages, pharmaceutical formulations thereof, and/or auxiliary agent(s) described herein can be administered in an amount ranging from about 0.01 μM to about 10 μM per day.

The nanocages or pharmaceutical formulations thereof described herein can be administered in combination with one or more other auxiliary agents that are independent of the pharmaceutical formulation. Suitable auxiliary agents include, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. Suitable compounds for the auxiliary active agents have been previously described in relation to the nanocages. The compound(s), and/or formulation(s), and/or additional therapeutic agent(s) can be administered simultaneously or sequentially by any convenient route in separate or combined pharmaceutical formulations. The additional therapeutic agents can be provided in their optically pure form or a pharmaceutically acceptable salt thereof.

In some embodiments, the nanocages are used to facilitate delivery of genome editing molecules. In further embodiments, a cell or population of cells can be incubated for period of time with one or more nanocages or formulations thereof described herein. In some embodiment, the nanocage contains a Cas9:sgRNA complex. The incubation time can range from about 1 h to about 10 days or more. Subsequent to incubation, the cell or cells can be cultured using techniques known in the art and/or described herein. The cell or cells can also be analyzed for genome modification using techniques and/or methods known in the art or as described herein.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

Self-assembled DNA nanostructures have been developed with precisely controlled size and architecture.¹ Because of DNA's intrinsic biocompatibility and degradability, DNA nanostructures hold tremendous promise for drug delivery. Numerous cargoes, including small-molecule drugs,² small interfering RNA (siRNA),^{1a,3} the immunostimulatory oligonucleotide CpG,⁴ photosensitizers,⁵ and proteins,^{1b} have been successfully delivered intracellularly by DNA nanocarriers. Moreover, DNA-based carriers can be readily functionalized either by hybridizing a targeting moiety onto the nanostructure1a or programming a targeting aptamer into the DNA chain^{1b,c} for targeted drug delivery. Despite these advances, strategies utilizing DNA scaffolds for on-demand drug delivery in a stimuli-responsive fashion,⁶ instead of passive release,⁷ still remain elusive. We have recently reported an adenosine triphosphate (ATP)-responsive formulation incorporating short DNA strands (with ATP's aptamer) loaded with doxorubicin (DOX), an anticancer drug.⁸ The enhanced drug release inside cancer cells triggered by a high ATP level was validated. However, this design is limited by a complicated formulation process and relatively low drug loading capacity.

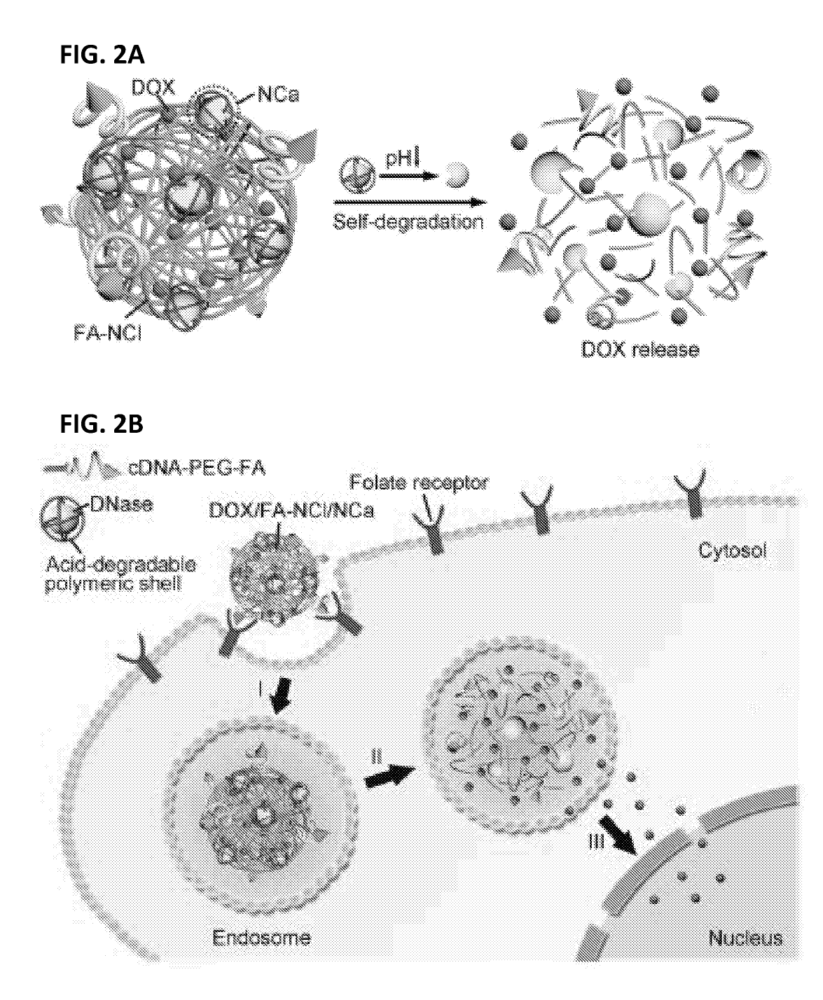

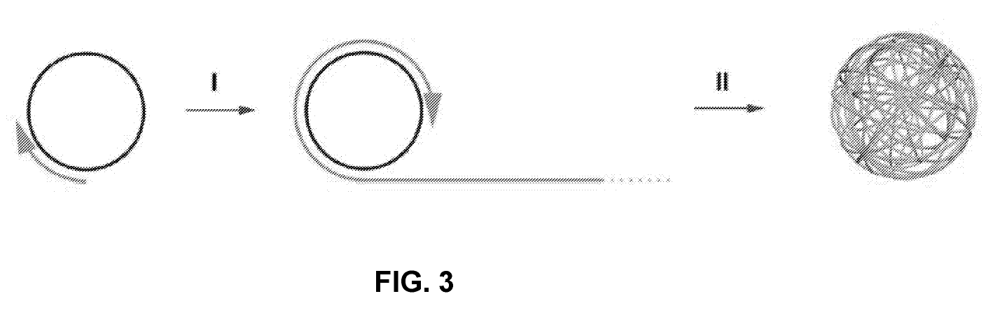

Described herein is a bioinspired drug delivery carrier in which a cocoon-like DNA nanocomposite is integrated with “caged worm” deoxyribonuclease (DNase) to achieve selfdegradation for promoting drug release inside cells (FIGS. 2A and 2B). The DNA structure is based on a “nanoclew” (denoted as NCI) that is “woven” by rolling-circle amplification (RCA) (FIG. 3), the product of which can be applied in biodetection.⁹ Multiple GC-pair sequences were integrated into the NCI to enhance the loading capacity of DOX.⁸ To facilitate self-assembly, a palindromic sequence was incorporated into the template. To enable degradation of NCI, DNase I was encapsulated into a single-protein-based nanocapsule (denoted as NCa) with a positively charged thin polymeric shell that is cross-linked by acid-degradable crosslinkers using interfacial polymerization (FIG. 2A).¹⁰ Furthermore, to achieve tumor-targeting delivery of DOX, folic acid (FA) was conjugated to an NCI complementary DNA (cDNA) oligomer followed by hybridization to the DNA NCI. The positively charged NCa can be embedded into the NCI via electrostatic interactions to form the DOX-loaded selfdegradable DNA scaffold (designated as DOX/FA-NCl/NCa). The polymeric capsule cages the activity of DNase I at physiological pH, causing DOX to be retained in the NCI. When DOX/FA-NCl/NCa is internalized by cancer cells and enters the acidic endolysosome, the polymeric shell of NCa degrades and is shed from DNase I. This results in the immediate rejuvenation of DNase I, which rapidly degrades NCI, thereby releasing the encapsulated DOX for enhanced anticancer efficacy (FIG. 2B). This formulation represents a stimuli-responsive drug delivery system, the trigger of which is preloaded with the delivery vehicle and can be activated by the cellular environment.

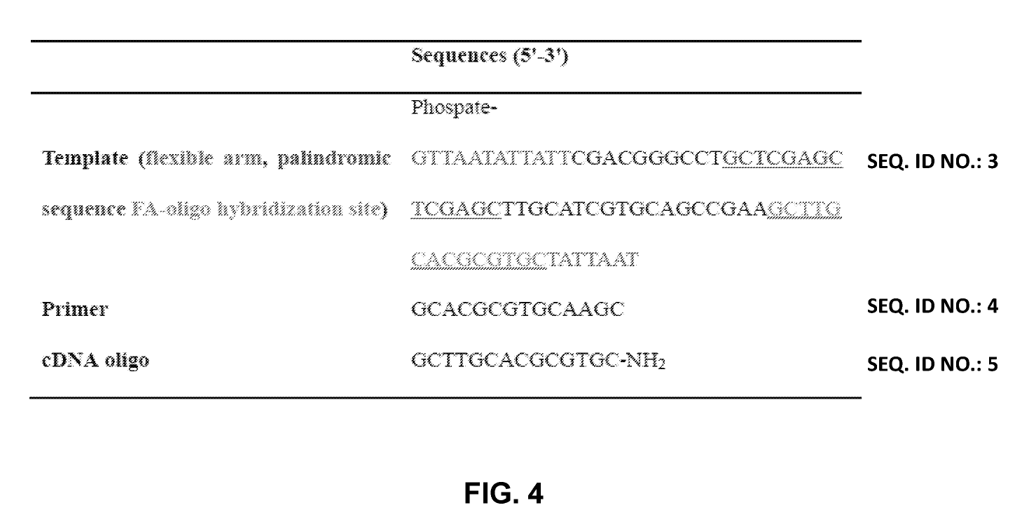

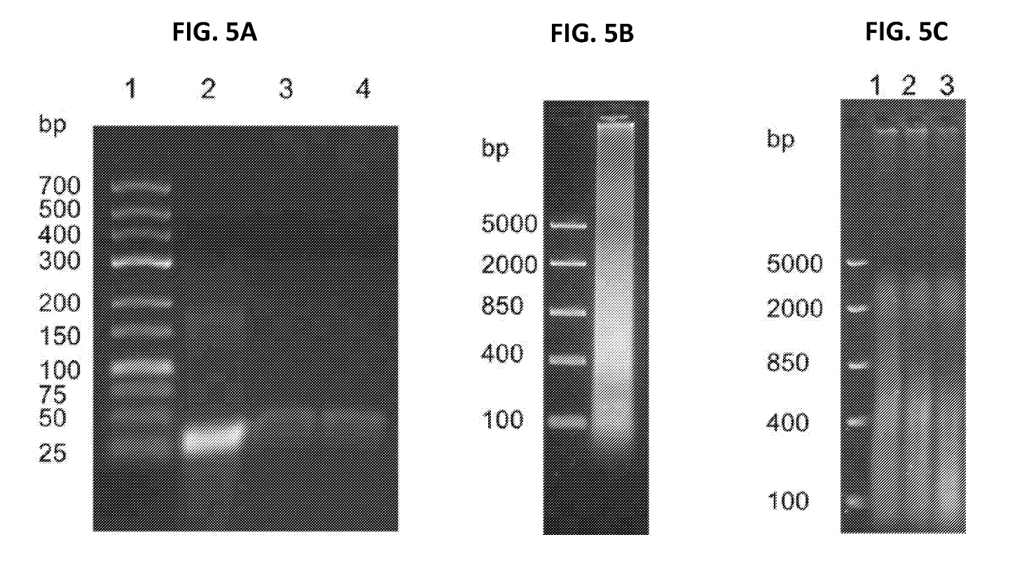

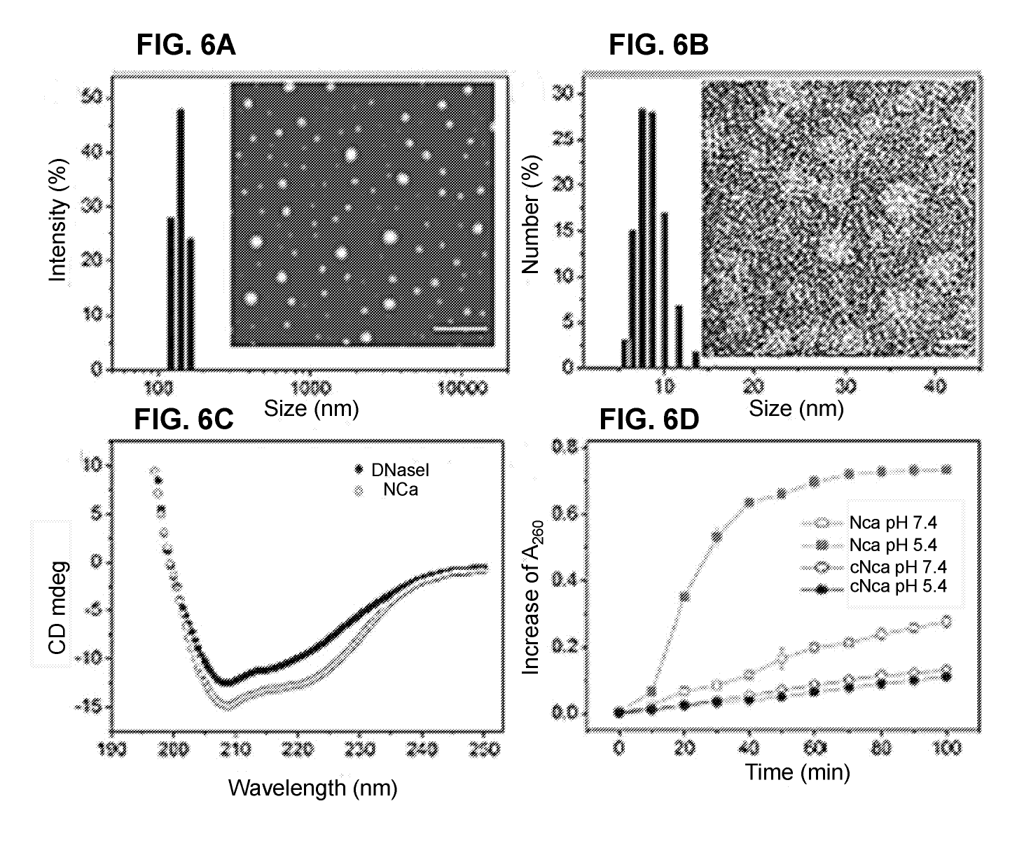

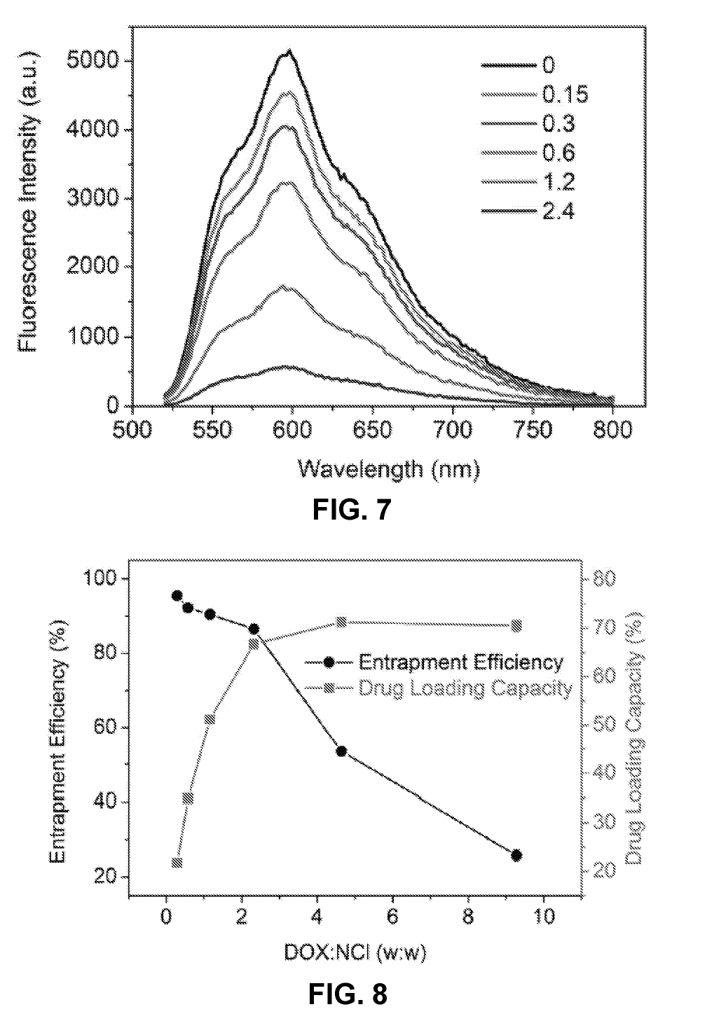

To validate our assumption, the DNA NCI by RCA was first synthesized (the sequence is shown in FIG. 4). Cyclization of the single-stranded DNA (ssDNA) template was confirmed by its resistance to Exonuclease I, and RCA products with various molecular weights were amplified from the circular ssDNA template (FIGS. 5A-5C). NCI exhibited high stability after incubation with culture medium containing fetal bovine serum (FBS) (10% v/v) for up to 48 h (FIG. 5C). The synthesized ssDNA self-assembled into the three-dimensional clew-like structure with an average particle size of 150 nm (FIG. 6A). Intercalation of DOX into NCI was monitored via the fluorescence intensity of the DOX solution, which significantly declined when NCI was added as a result of selfquenching⁸ of DOX upon interacting with the NCI (FIG. 7). The DOX loading was also assessed (FIG. 8). It was found that at a mass ratio of 2.3, NCI showed a maximum DOX loading capacity of 66.7%, and 86.5% of the added DOX was entrapped in the obtained NCI.

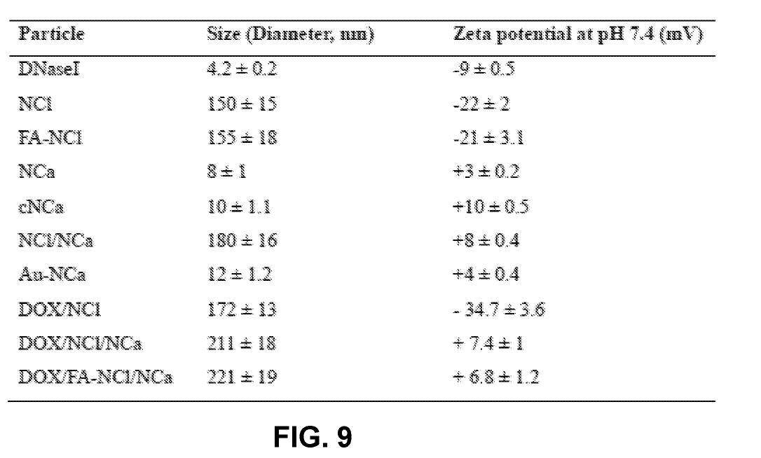

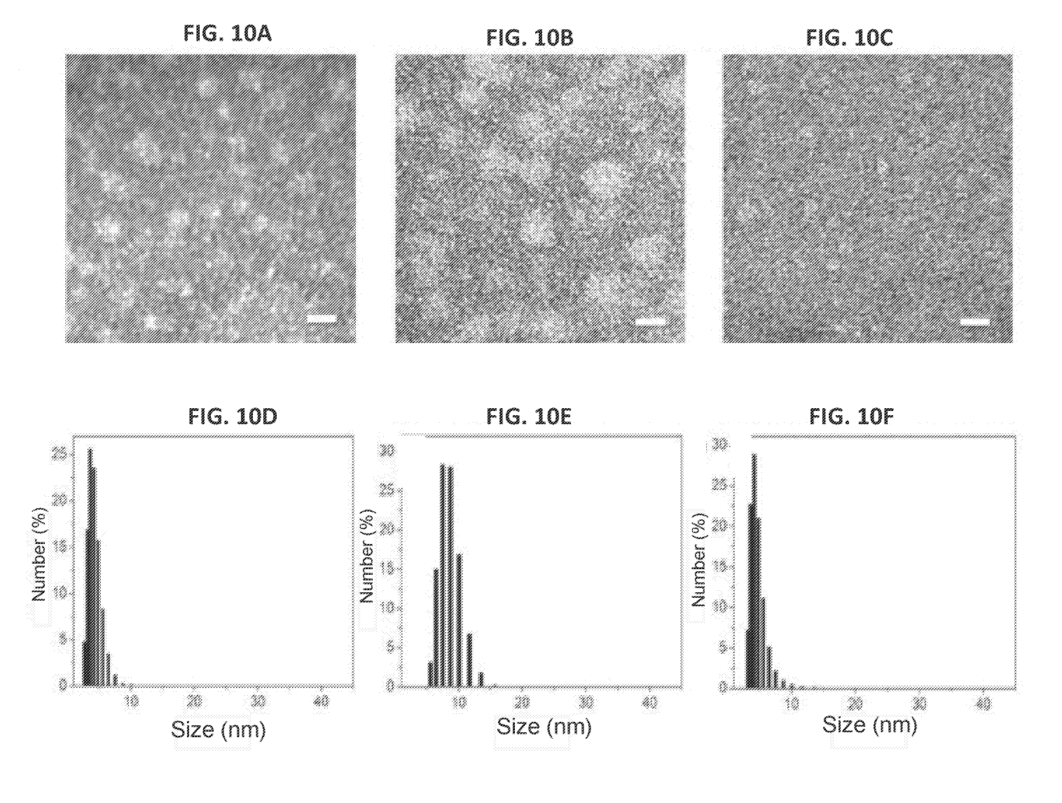

Both native DNase I and the obtained NCI had negatively charged surfaces (FIG. 9). To integrate them together, DNase I was encapsulated in a positively charged polymeric single-protein nanogel by means of in situ free-radical polymerization,^{10b} which encapsulated DNase I into a capsule with the ζ potential converted from −9 to +3 mV. Monodispersed NCa was obtained with an average particle size of 8.0 nm, which is larger than the size of the native DNase I (4.2 nm) (FIG. 6B). Encapsulating DNase I in the capsule had no impact on its secondary structure (FIG. 6C), and acid responsive degradation¹¹ of NCa was observed (FIGS. 10A-10F). Glycerol dimethacrylate (GDA), the pH-responsive cross-linker in NCa, is stable at physiological pH but degradable at a lower pH,^{10a} NCa degradation was observed after incubation at pH 5.4 for 2 h. The particle size of NCa was remarkably decreased at pH 5.4 compared with that at pH 7.4.

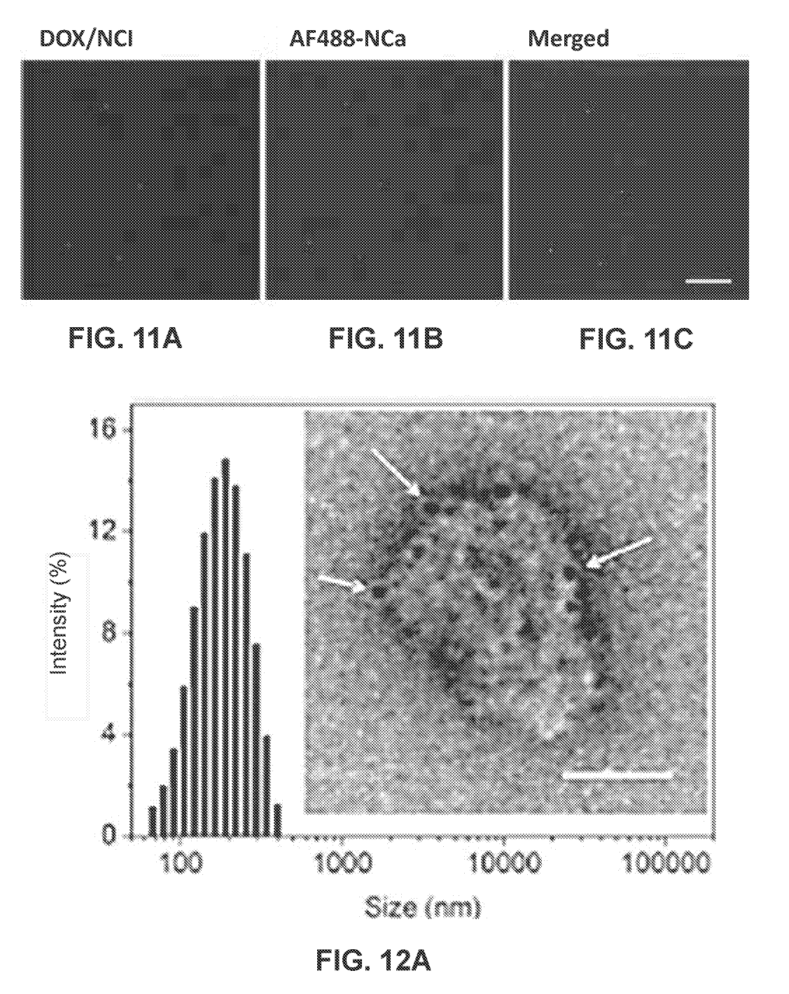

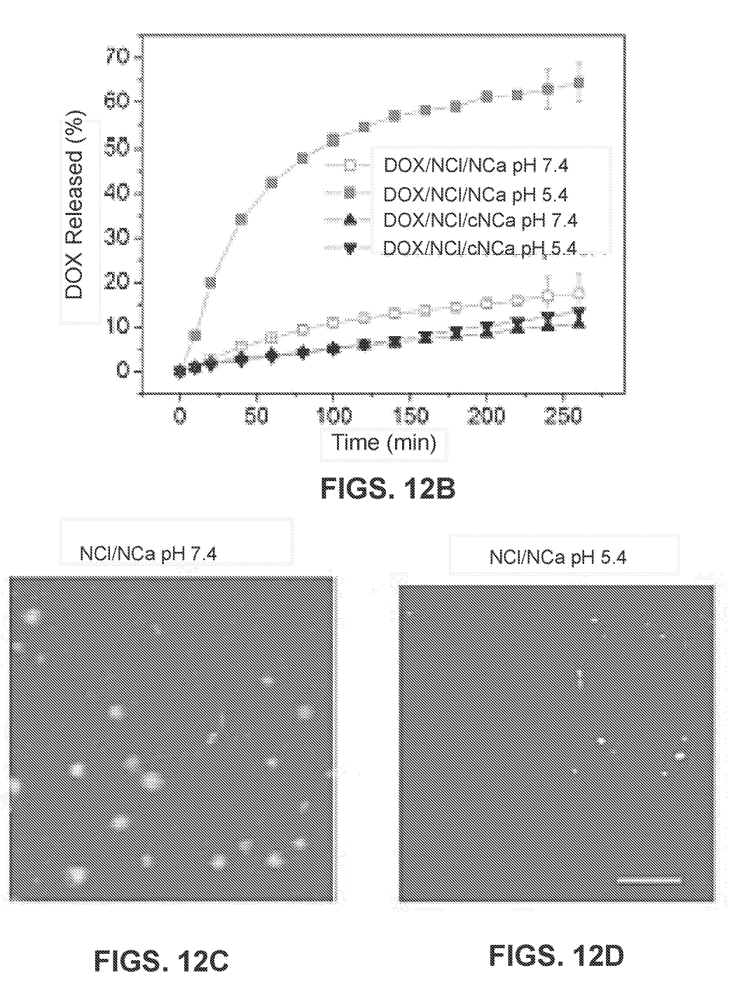

To further substantiate the pH-responsive DNA-degrading capability of NCa, a nondegradable DNase I capsule (cNCa) prepared with a nondegradable cross-linker, methylenebis-(acrylamide), in place of GDA was used as a control. The pH responsiveness of NCa was further confirmed by testing the enzymatic activity of DNase I (FIG. 6D). Because of the nondegradability of cNCa, the polymeric shell of cNCa impeded the DNase I activity at both pH 7.4 and 5.4. However, NCa showed significantly higher DNase I activity at pH 5.4 than that at pH 7.4. Then negatively charged NCI was mixed with positively charged NCa to form homogeneous NCl/NCa complexes (PDI=0.24±0.02). The NCl/NCa assembly was observed by the colocalization of the fluorescence signals of DOX (red) in DOX/NCI and Alexa Fluor 488 (AF488) (green) in AF488-modified NCa (FIGS. 11A-11C). The NCl/NCa assembly increased the average hydrodynamic size of NCI from 150 to 180 nm, and the NCI ζ potential was converted from negative to positive (FIG. 12A and FIG. 9). Furthermore, the TEM image clearly showed that gold nanoparticle-labeled NCa^{10a,12} (Au-NCa) (FIG. 9) was well-decorated onto the NCI surface (FIG. 12A).

The release profiles of DOX from DOX/NCl/NCa at different pH values were determined^{8a} (FIG. 12B), and pH reduction resulted in promoted release of DOX. At pH 5.4, the cumulative release of DOX within 260 min was 3.7-fold that at pH 7.4. In contrast, there was no apparent difference in the release of DOX from DOX/NCI/cNCa at pH 5.4 and 7.4. Similarly, The NCl/NCa complexes remained stable at pH 7.4 for 2 h, while a high degradation efficiency of NCl/NCa complexes was observed at pH 5.4 (FIGS. 12C-12D).

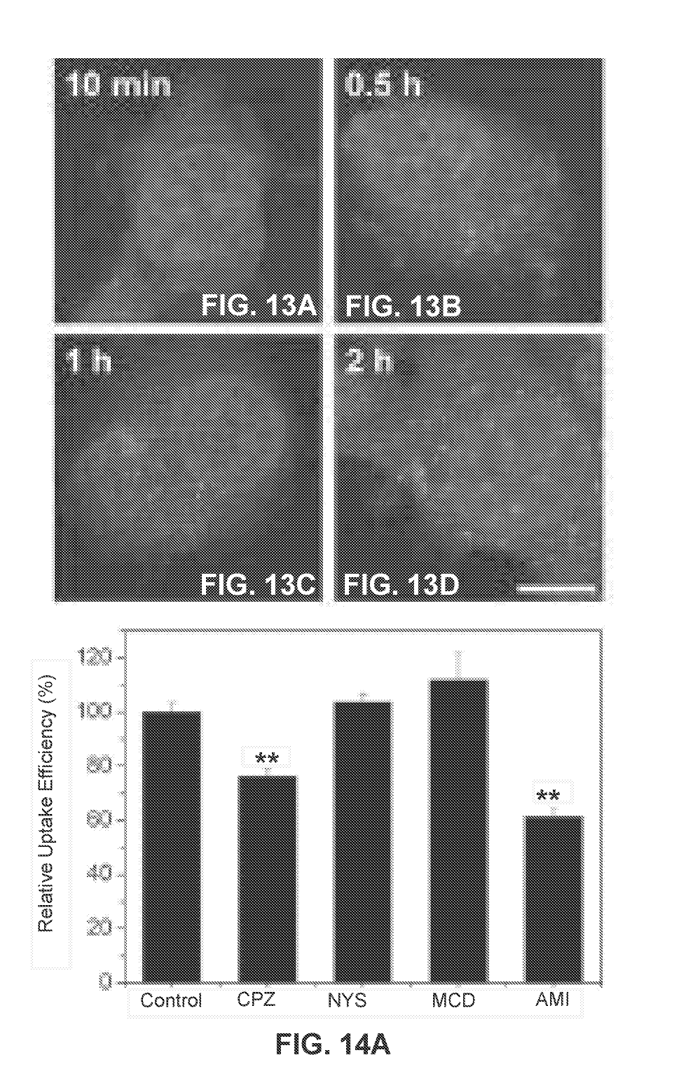

To enhance the tumor-targeting efficacy of DOX/NCl/NCa, a ligand containing FA (cDNA-PEG-FA) was hybridized into the NCI, and the hybridization of cDNA-PEG-FA to the NCI resulted in no significant change in the NCI particle size and ζ potential (FIG. 9). The endocytosis pathway of DOX/FANCl/NCa was determined by incubating human breast cancer (MCF-7) cells overexpressing FR13 with different inhibitors for specific pathways (FIGS. 13A-13D). Compared with other inhibitors, both chlorpromazine (CPZ) and amiloride (AMI) displayed pronounced effects in inhibiting the internalization of DOX/FA-NCl/NCa, suggesting that DOX/FA-NCl/NCa was internalized by the cells and localized in the acidic endosomes.

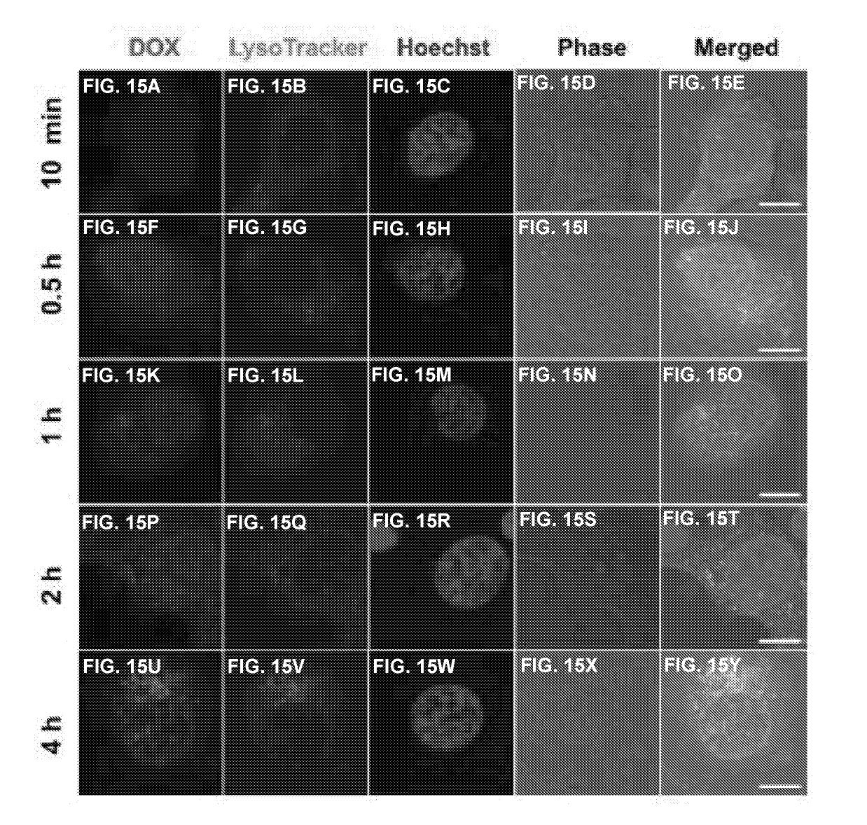

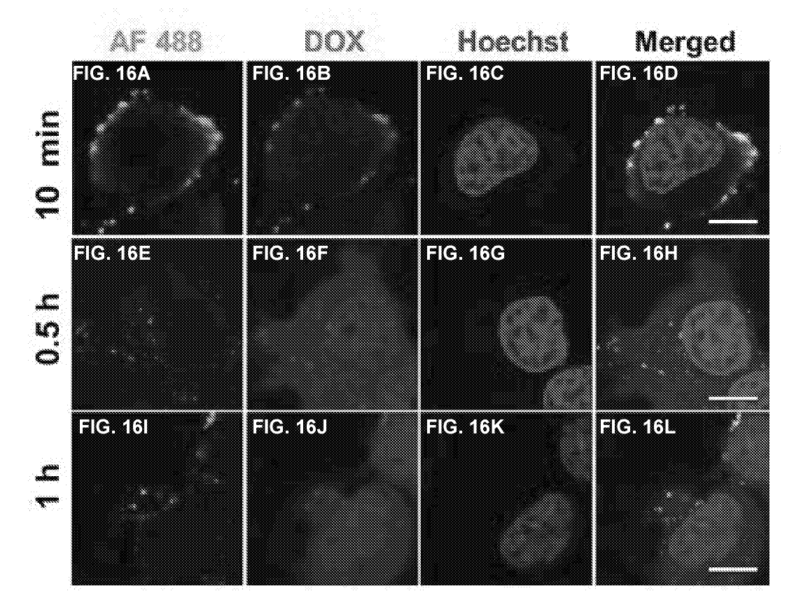

The intracellular distribution of DOX/FA-NCl/NCa was then detected (FIGS. 13A-13D and 15A-15Y). The internalization and nucleus targeting of DOX/FA-NCl/NCa in MCF-7 cells was extremely fast even within the first 10-30 min, during which period obvious endolysosomal entrapment and nucleus targeting of DOX could be observed. Colocalization of DOX/FA-NCI with NCa in MCF-7 cells was also observed (FIGS. 16A-16L). In the first 10 min, DOX/FA NCI/AF488-NCa was internalized together. The fluorescence signals of DOX and AF488 showed a high colocalization. After 0.5 h, a large amount of DOX was released from the DOX/FA-NCI/AF488-NCa into the cytosol and specifically accumulated in the nucleus. Such rapid cytosolic distribution and nucleus-targeting effects of DOX delivered by DOX/FA-NCl/NCa can be attributed to the efficient degradation of DOX/FA-NCI by NCa to promote the release of DOX.

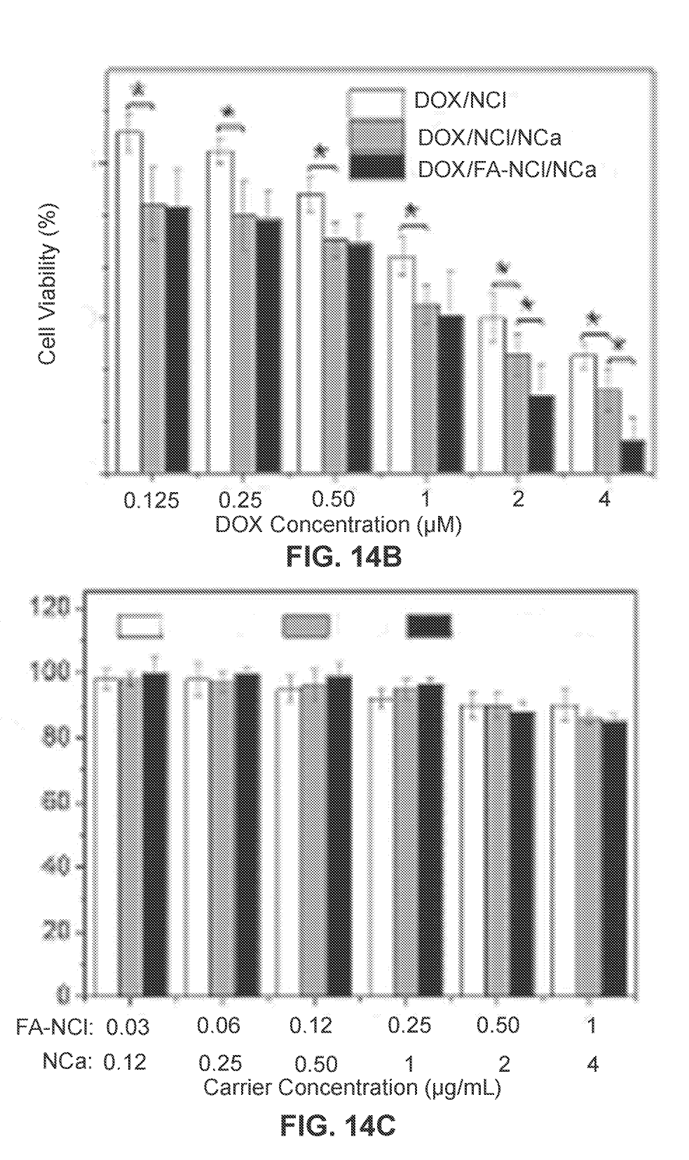

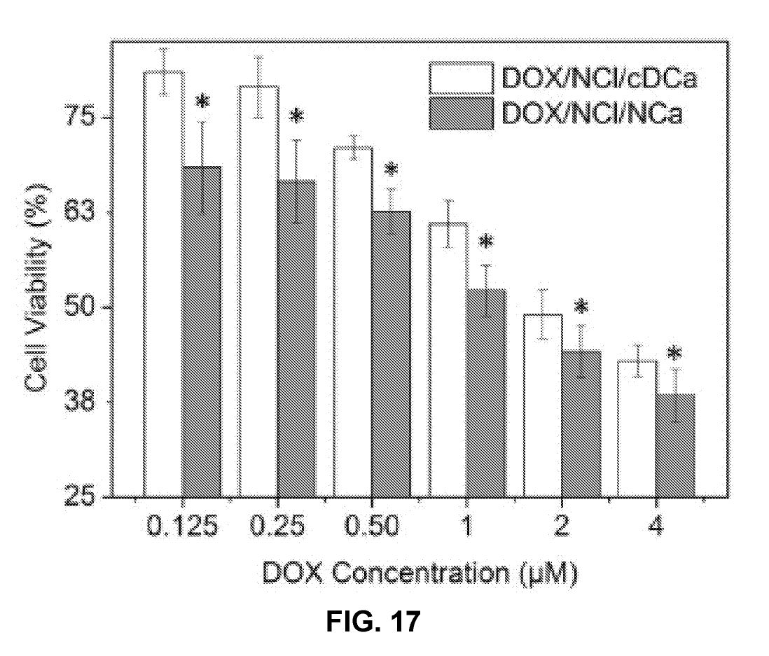

The in vitro cytotoxicities of DOX/NCI, DOX/NCl/NCa, and DOX/FA-NCl/NCa against MCF-7 cells were estimated (FIG. 14B). DOX/NCl/NCa showed a remarkably higher cytotoxicity toward MCF-7 cells than DOX/NCI. The halfmaximal inhibitory concentration (IC50) of DOX/NCl/NCa was calculated to be 1.2 μM, which is noticeably lower than the value of 2.3 μM for DOX/NCI. This verified that the NCamediated DOX release increased the toxicity of DOX delivered by NCI. This was further validated by the significantly higher cytotoxicity of MCF-7 treated with DOX/NCl/NCa than that associated with DOX/NCI/cNCa (FIG. 17). Additionally, the conjugation of FA onto the NCI surface enhanced the therapeutic efficacy of DOX (FIG. 14B). DOX/FA-NCl/NCa had the lowest IC50 (0.9 μM) compared with both DOX/NCl/NCa and DOX/NCI. The blank FA-NCI without DOX showed negligible toxicity at all tested concentrations (FIG. 14C). Although DNase I, the component of the carrier in this research, has been used as an anticancer agent in some other studies,¹⁴ the cytotoxicity of NCa toward MCF-7 at the selected concentration in this study was compromised compared with that of released DOX (FIG. 14C). FIG. 14A demonstrates the relative uptake efficiency of DOX/FA-Ncl/NCa by MCF-7 cells.

In summary, a bioinspired self-degradable drug delivery system containing a woven DNA “nanoclew” as a “cocoon matrix” and a “caged” DNase I nanogel as “hibernating worms” has been developed. The “worms” can be readily activated to degrade their cocoon to release encapsulated drugs in the endolysosomal compartments. This strategy provides insights for the design of new prodrugs and can be further extended to engineer other programmed drug delivery systems.

Materials and Methods

Materials.

All chemicals were purchased from Sigma-Aldrich unless otherwise specified, and were used as received. Doxorubicin was purchased from BIOTANG Inc. (Lexington, Mass., USA). DNA oligos were purchased form Integrated DNA Technologies Inc. (Coralville, Iowa, USA). Bovine pancreas DNase I lyophilized powder was purchase from Roche Applied Science (Mannheim, Germany). CircLigase II ssDNA Ligase was purchased from Epicenter (Madison, Wis., USA). Bst 2.0 DNA polymerase was purchased from New England BioLabs Inc. (Ipswich, Mass., USA). DNA ladder, dNTP, and Exonuclease I were purchased from ThermoFihser Scientific, Inc. (Pittsburgh, Pa., USA). Alexa Fluor® 488 Nhydroxysuccinimidyl ester was purchased from Life Technologies (Grand Island, N.Y., USA). Folic acidpolyethylene glycol 2000-N-hydroxysuccinimidyl ester (FA-PEG-NHS) was purchased from Nanocs Inc. (New York, N.Y., USA). Glycerol dimethacrylate was purchased from Tokyo Chemical Industry Co., Ltd. (Tokyo, Japan). GelRed was purchased from Biotium Inc. (Hayward, Calif., USA). Mono-sulfo-Nhydroxy-succinimido Au-nanoparticles was purchased from NanoProbe (Yaphank, N.Y., USA).

Preparation and Characterization of DNA nanoclew (NC/). Rolling circle amplification (RCA) was used to prepare the NCI. A 5′-phosphorylated ssDNA template (FIG. 4) was cyclized into a circular ssDNA template with CircLigase II ssDNA ligase according to manufacture's instructions. Briefly, 10 pmol ssDNA template was added into a 20 μL of reaction mixture containing 2.5 mM MnCl2, 1 M betaine and 5 U/μL CircLigase II ssDNA ligase. After incubation at 60° C. for 1 h, the cyclized template was treated with Exonuclease I (1 U/μL) at 37° C. for 45 min and followed by heat inactivation at 80° C. for 15 min. The resultant cyclized ssDNA template was hybridized with 0.5 μM primer in a 1× isothermal amplification buffer (20 mM Tris-HCl pH 8.8, 10 mM (NH4)2SO4, 50 mM KCl, 2 mM MgSO4, 0.1% Tween 20) containing 0.2 mM dNTP at 95° C. for 5 min. After cooling the template-primer hybridized solution to room temperature, Bst 2.0 DNA polymerase was added to a final concentration of 0.2 U/μL and the RCA was performed at 60° C. for 17 h followed by heat inactivation at 80° C. for 20 min. The obtained NCI was dialyzed against TE buffer (10 mM Tris pH 8.0, 1 mM EDTA) at room temperature in a dialysis unit (20 K MWCO) (Slide-A-Lyzer, Thermo Scientific) for 48 h. Sizes of the template and product were estimated by agarose gel electrophoresis using 0.8% (w/v) agarose gel. After staining with GelRed, the agarose gel was imaged under UV irradiation. DNA concentration of NCI was measured with Nanodrop 2000C spectrometer (Thermo Scientific). Particle size and zeta potential were measured by a Zetasizer (Nano ZS, Malvern). Stability of the RCA product was tested by incubating it in DMEM media containing 10% fetus bovine serum (FBS) at 37° C. for 48 h. For atomic force microscope, NCI was dropped onto a silicon wafer (Ted Pella), dried and analyzed with Nanoscope (Veeco, Santa Barbara, Calif.) using tapping mode in ambient air.

Preparation and Characterization of DNase I Nanocapsule (NCa).

DNase I was encapsulated in a single protein nanocapsule cross-linked by pH responsive cross-linker.¹⁵ DNase I lyophilized powder was dissolved in 5 mM bicarbonate buffer (pH 8.3) to 1 mg/mL with a PDI of 0.38±0.03 as determined by Zetasizer (Nano ZS, Malvern). 200 mg/mL acrylamide (AAm) was added to 1 mL of DNase I solution while stirring at 4° C. After 10 min, the positively charged monomer N-(3-aminopropyl) methacrylamide (APMAAm) was added while stirring. Then the pH responsive cross-linker glycerol dimethacrylate (GDA) was added together with 30 μL ammonium persulfate (100 mg/mL in deoxygenated and deionized water) and 3 μL N,N,N′,N′-tetramethylethylenediamine to initiate the polymerization. The molar ratio of AAm/APMAAm/GDA was 10/1/0.13. The polymerization was allowed to proceed for 60 min and then buffer changed with phosphate-buffered saline (pH 7.4) to remove excess monomers and initiators in a ultracentrifuge unit (MWCO 30 KDa, Millipore). Protein content in the NCa was determined by bicinchoninic acid (BCA) colorimetric protein assay (Thermo Scientific) with bovine serum albumin (BSA) as the standard. Far-UV circular dichroism (CD) spectra of native DNase I and NCa was performed at 20° C. in 0.1 M phosphate buffer (pH 7.4) with protein concentration of 0.2 mg/mL (JASCO J-815 Circular Dichroism Spectrometer). Size and zeta potential of native DNase I and NCa were determined by a Zetasizer (Nano ZS, Malvern). For the non-degradable NCa, a non-degradable crosslinker methylenebisacrylamide was used instead of GDA while other conditions remained the same. For TEM imaging, the samples were dropped on a TEM copper grid (300 mesh) (Ted Pella) and stained with 2% (w/v) uranyl acetate (dissolved in 50% ethanol). The samples were observed by TEM (JEM-2000FX, Hitachi) at 100 kV. DNase I activity was assayed with DNA sodium salt from salmon (0.2 mg/mL) as the substrate at 37° C. in 200 mM phosphate buffer containing 2.5 mM MgCl2 and 0.5 mM CaCl2. A260 increases over time were recorded by Nanodrop 2000C (Thermo Scientific).

DOX Loading and Release.

The capability of NCI to load DOX was tested by measuring its ability to quench the fluorescence of DOX. NCI of different final concentrations (0.15-2.4 μg/mL) was added to 10 μM DOX solution and the fluorescence of DOX/NCI was scanned (excitation 480 nm, emission 520-800 nm) in a microplate reader (Infinite M200 PRO, Tecan). To test the DOX loading efficiency of NCI, 10 μg/mL NCI was incubated with different concentrations of DOX (5-160 μM, DOX/NCI mass ratio of 0.3-9.3) at room temperature for 1 h. The mixture was then centrifuged at 14000×g for 10 min and DOX concentration in the supernatant was analyzed by reading DOX fluorescence (excitation 480 nm, emission 596 nm). Entrapped DOX was calculated as DOX added in the beginning—DOX in the supernatant after centrifugation, DOX entrapment efficiency is calculated as mass of DOX entrapped/mass of DOX added. DOX loading capacity of the DNA carrier is calculated as mass of DOX entrapped/(mass of DOX entrapped+mass of carrier). DOX release profile from NCI was monitored by measuring DOX fluorescence recovery from DOX/NCl/NCa or DOX/NCI/cNCa, which was incubated in 0.2 M phosphate buffer (pH 7.4, 5.4) containing 2.5 mM MgCl2 and 0.5 mM CaCl2 at 37° C. for 260 min. Samples were taken and centrifuged at 14000×g for 10 min and DOX released was quantified by measuring DOX fluorescence.

Assembly and Characterization of NCl/NCa.

10 μg/mL NCI was mixed with 40 μg/mL NCa before each use. Size and zeta potential of NCl/NCa was measured by a Zetasizer (Nano ZS, Malvern). To visualize the assembly by confocal laser scanning microscope (CLSM), DNase I was conjugated with Alexa Fluor® 488 N-hydroxysuccinimidyl ester (AF488-NHS) to obtained AF488 decorated DNase I (AF488-DNase I). 1 mg DNase I was dissolved in 1 mL 0.1 M bicarbonate buffer (pH 8.3), equimolar amount of AF488-NHS (dissolved in anhydrous DMSO) was added into DNase I solution while stirring.

The reaction was kept at room temperature for 1 h and excessive AF488-NHS was removed by ultracentrifugation (30 K MWCO, Millipore). AF488-DNase I was encapsulated in acid degradable protein capsule by the same method as described above to obtain AF488-NCa. NCI was visualized by the fluorescence of the loaded DOX. The DOX/NCI/AF488-NCa was immobilized in 1% agarose gel and observed with CLSM (LSM 710, Zeiss). To visualize NCl/NCa by TEM, gold nanoparticle (AuNP) was conjugated onto DNase I for imaging.¹⁵⁻¹⁶ Mono-sulfo-N-hydroxy-succinimido AuNP was reacted with native DNase I at molar ratio of 4/1 in PBS buffer (pH 7.4) for 1 h. Excessive AuNP was removed by gel filtration using Superdex-75. Concentrations of the AuNP and DNase I was determined by UV/vis spectra based on the molar extinction coefficients (AuNP, 155,000 M-1 cm-1 at 420 nm, DNase I, 36750 M-1 cm-1 at 280 nm). The resulted Au-DNase I had a molar ratio of AuNP/DNase I of 0.91 and it was encapsulated in the nanocapsule (Au-NCa) by the same method as native DNase I. The obtained Au-NCa was mixed with NCI and dropped onto a TEM grid followed by rinsing with deionized water. Silver enhancement was applied for better TEM imaging by floating the grid on fresh silver enhancement reagent (Nanoprobe) for 1 min. Then the grid was rinsed with deionized water and stained with 1% sodium phosphotungstate at pH 7.0. Uniformed 3-4 nm silver coated AuNP form by this process. PH triggered NCl/NCa degradation was visualized by AFM with NCl/NCa samples incubated at pH 7.4 and pH 5.4 phosphate buffer for 2 h before imaging.

Conjugation of NCl with Folic Acid.

The complementary DNA (cDNA) oligo (Table S1) for folic acid conjugation was modified with NH2 at 3′ end. After dissolving the oligo in 0.1 M bicarbonate buffer (pH 8.3), folic acid-polyethylene glycol 2000-N-hydroxysuccinimidyl ester (FA-PEG-NHS, dissolved in DMSO) was added while stirring with molar ratio of FA-PEG-NHS/oligo at 2/1. The reaction was protected from light and allowed to proceed at room temperature overnight. The conjugation product (cDNA-PEG-FA) was dialyzed against deionized water in a dialysis unit (MWCO 3.5K, Millipore) for 48 h. The obtained cDNA-PEG-FA was hybridized with the NCI at 95° C. for 5 min and then cooled to room temperature. Molar ratio of the repeating unit in NCI to cDNA-PEG-FA was 100:1. Size and zeta potential of FA-NCI was measured by a Zetasizer (Nano ZS, Malvern).

Cell Culture.

Human breast cancer cell line MCF-7 from American Type Culture Collection (Manassas, Va.) was cultured in Dulbecco's Modified Eagle's Medium supplemented with 10% (v/v) FBS, penicillin (100 U/mL) and streptomycin (100 μg/mL) in a 37° C. incubator (Thermal Scientific) under 5% CO2 and 90% humidity. The cells were regularly sub-cultured with trypsin-EDTA (0.25%, w/w) and cell density was determined with hemocytometer before each experiment.

Determination of Endocytosis Pathways.

MCF-7 cells were seeded in 6-well plates (1×104 cells per well) and culture for 48 h. Afterwards, the cells were pre-incubated with several inhibitors specific for different endocytosis pathways¹⁷ [chlorpromazine (CPZ, 10 μM) for clathrin-mediated endocytosis; nystatin (NYS, 25 μg/mL) for cavelolin-mediated endocytosis; amiloride (AMI, 1 mM) for macropinocytosis; methyl-β-cyclodextrin (MCD, 3 mM) for lipid raft] for 1 h at 37° C., respectively. Then the cells were incubated with DOX/FA-NCl/NCa at DOX concentration of 2 μM or FA-NCI/AF488-NCa of the same concentration in the presence of the inhibitors for another 2 h. After washing the cells by 4° C. PBS twice, the cells were lysed with Pierce IP lysis buffer (Thermo Scientific) and centrifuged. Fluorescence of DOX and AF488 and protein concentration in the supernatant were measured, respectively, by a microplate reader (Infinite M200 PRO, Tecan).

Intracellular Distribution.

MCF-7 cells (1×105 cells per dish) were seeded in confocal dishes and cultured for 24 h. To image nucleus targeting by DOX, the cells were incubated with DOX/FA-NCl/NCa at DOX concentration of 2 μM for 10 min, 0.5 h, 1 h, 2 h and 4 h. Afterwards, the cells were washed with 4° C. PBS twice and stained by LysoTrcker green (50 nM) (Life Technologies) at 37° C. for 30 min. Then the cells were washed with 4° C. PBS twice and stained with Hoechst 33342 (1 μg/mL) for 10 min. After washing 4° C. PBS twice again the cells were immediately observed on CLSM (LSM 710, Zeiss). On the other hand, to image the co-localization of NCI and NCa, the cells were incubated with DOX/FANCl/AF488-NCa at DOX concentration of 2 μM for 10 min, 0.5 h and 1 h. The cells were then washed with 4° C. PBS twice and stained with Hoechst 33342 (1 μg/mL) for 10 min. After washing 4° C. PBS twice again the cells were immediately observed on CLSM.

In Vitro Cytotoxicity Assay.

MCF-7 cells were seeded in 96-well plate (1×104 cells per well). After culturing for 24 h, the cells were exposed to DOX/FA-NCl/NCa at different DOX concentrations in FBS free medium for additional 24 h. Afterwards, 20 μL per well of MTT solution (5 mg/mL) was added and incubate for another 4 h. After removing the culture media, the cells were mixed with 150 μL of DMSO. The absorbance was measured at a test wavelength of 570 nm with a reference wavelength of 630 nm by a microplate reader (Infinite M200 PRO, Tecan).

### REFERENCES FOR EXAMPLE 1

(1) (a) Lee, H. et al. Nat. Nanotechnol. 2012, 7, 389-393. (b) Douglas, S. M. et al. Science 2012, 335, 831-834. (c) Andersen, E. S. et al. Nature 2009, 459, 73-76. (d) Zhang, Z.; et al. ChemBioChem 2014, 15, 1268-1273. (e) Lo, P. K.; et al. Nat. Chem. 2010, 2, 319-328. (f) Zhang, Z. et al. Biomaterials 2013, 34, 9728-9735. (g) Zhu, G.; et al. Am. Chem. Soc. 2013, 135, 16438-16445. (h) Hu, R.; et al. Angew. Chem., Int. Ed. 2014, 53, 5821-5826.
(2) (a) Kim, K.-R. et al. Chem. Commun. 2013, 49, 2010-2012. (b) Zhao, Y.-X. et al. ACS Nano 2012, 6, 8684-8691.
(3) Lee, J. B. et al. Nat. Mater. 2012, 11, 316-322.
(4) (a) Schuller, V. J. et al. ACS Nano 2011, 5, 9696-9702. (b) Li, J. et al. ACS Nano 2011, 5, 8783-8789.
(5) Wang, K.; et al. Angew. Chem., Int. Ed. 2011, 50, 6098-6101.
(6) (a) Xiao, Z. et al. Angew. Chem., Int. Ed. 2012, 51, 11853-11857. (b) Zhang, Z. et al. Am. Chem. Soc. 2010, 132, 14113-14117.
(7) (a) Peer, D. et al. Nat. Nanotechnol. 2007, 2, 751-760. (b) Shi, J.; et al. Nano Lett. 2010, 10, 3223-3230. (c) Chow, E. K.-H.; Ho, D. Sci. Transl. Med. 2013, 5, No. 216rv214.
(8) (a) Mo, R. et al. Nat. Commun. 2014, 5, 3364. (b) Mo, R.; Jiang, T.; Gu, Z. Angew. Chem., Int. Ed. 2014, 53, 5925-5930.
(9) Ali, M. M. et al. Chem. Soc. Rev. 2014, 43, 3324-3341.
(10) (a) Yan, M. et al. Nat. Nanotechnol. 2010, 5, 48-53. (b) Gu, Z.; et al. Nano Lett. 2009, 9, 4533-4538.
(11) (a) Murthy, N.; et al. J. Am. Chem. Soc. 2002, 124, 12398-12399. (b) Murthy, N.; et al. J. Proc. Natl. Acad. Sci. U.S.A. 2003, 100, 4995-5000. (c) Bachelder, E. M.; et al. J. Am. Chem. Soc. 2008, 130, 10494-10495.
(12) Liu, Y. et al. Nat. Nanotechnol. 2013, 8, 187-192.
(13) Li, K. et al. Chem. Commun. 2011, 47, 7323-7325.
(14) Alcazar-Leyva, S. et al. Med. Sci. Monit. 2009, 15, CR51-CR55.
(15) M. Yan, et al. Nat. Nanotechnol. 2010, 5, 48-53.
(16) Y. Liu, et al. Nat. Nanotechnol. 2013, 8, 187-192.
(17) R. Mo, et al. Nat. Commun. 2014, 5, 3364.
(18) R. Drmanac, A. B. et al. Science 2010, 327, 78-81.

### Example 2

CRISPR-Cas9 has rapidly transitioned from an RNA-directed defense system in prokaryotes to a facile genome-editing technology.^{[1]} The editing merely requires the Cas9 nuclease and an engineered single-guide RNA (sgRNA): the 20-nucleotide guide portion of the sgRNA recognizes complementary DNA sequences flanked by a protospacer-adjacent motif (PAM), and Cas9 cleaves the recognized DNA.^{[2]} The double-stranded break is then repaired through non-homologous end joining (NHEJ) or homology-directed repair (HDR), allowing defined alterations to the targeted region.^{[3]}

As CRISPR-Cas9 systems undergo further development toward human therapeutics, delivery poses the major challenge. Cas9 and the sgRNA have been overwhelmingly encoded within the DNA of plasmids of viral vectors.^{[4]} However, this DNA can randomly integrate into the genome, potentially giving rise to cancer or other genetic diseases.^{[5]} Furthermore, the template-driven nature of gene expression limits control over the total amount of Cas9 protein and sgRNAs, where excess dosing has been attributed to off-target cleavage.^{[6]} One alternative is to deliver the Cas9/sgRNA ribonucleotprotein complex,^{[7]} which enables greater control over its intracellular concentration and limits the timeframe in which editing can occur. However, delivering protein and RNA remains a central challenge in drug delivery.^{[8]} Most protein therapeutics, such as enzymes,^{[9]} antibodies^{[10]} or transcription factors,^{[11]} suffer from low stability and poor cell membrane permeability as a result of their fragile tertiary structures and large molecular sizes.^{[8]} The strong negative charges of RNA therapeutics, including siRNA or miRNA, blocks them from diffusing across cell membrane and their susceptibility to endonuclease often requires chemical modification to prevent degradation.^{[12]} Thus, devising an appropriate carrier to shield the protein and RNA from detrimental physiological environment and escort them simultaneously to cell nucleus is highly desirable.

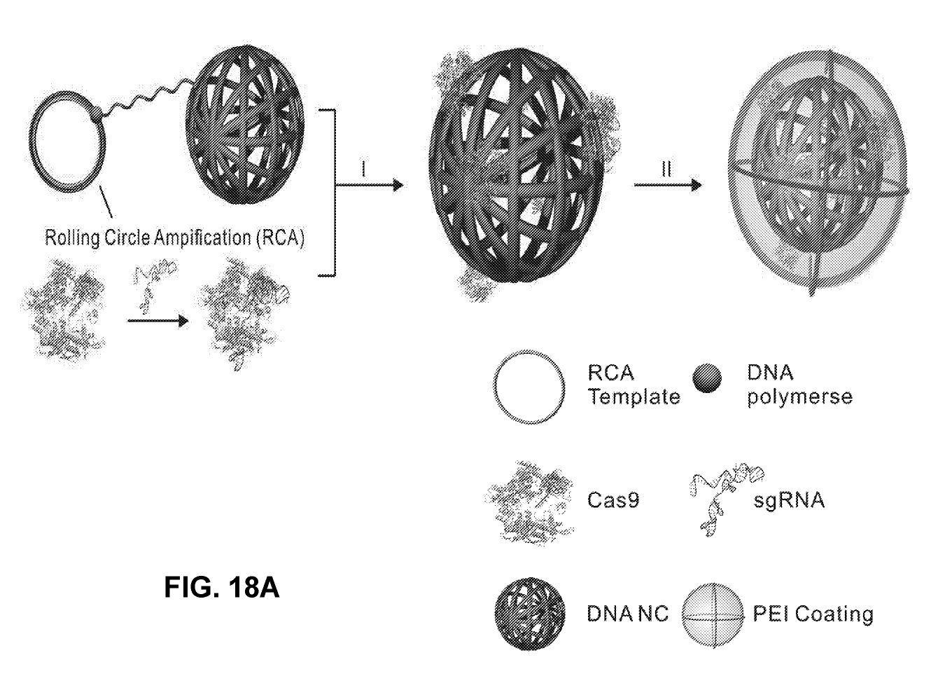

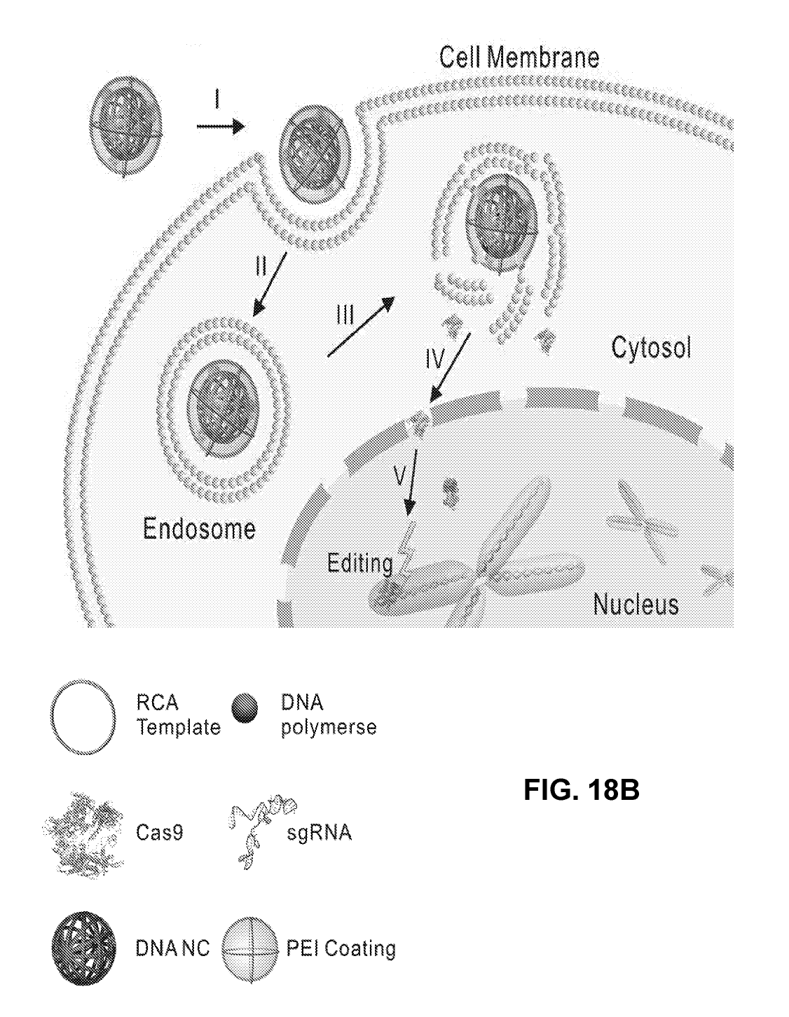

This Example demonstrates a delivery vehicle for CRISPR-Cas9 using a yarn-like DNA nanoclew (NC) (FIGS. 18A-18B). The DNA NCs can be synthesized by rolling circle amplification (RCA)^{[13]} with palindromic sequences encoded to drive the self-assembly of nanoparticles. It was previously demonstrated that the DNA NC could encapsulate the chemotherapeutic agent doxorubicin and drive its release based on environmental conditions.^{[14]} As demonstrated in this example, the DNA NC can load and deliver the Cas9 protein together with a sgRNA for genome editing. The DNA NC was configured to be partially complementary to the sgRNA such that the sgRNA can base pair with the ssDNA of the DNA NC^{[15]}. Following loading of the DNA NC with the Cas9/sgRNA complex, a coating of the cationic polymer polyethylenimine (PEI) was applied to help induce endosomal escape.^{[16]} The Cas9/sgRNA complex delivered to the cytoplasm could then be transported into the nucleus via nuclear-localization-signal peptides fused to Cas9. We expected that the resulting delivery vehicle could form uniform particles and drive the formation of targeted insertions or deletions (indels) without measurable impact on cell viability.

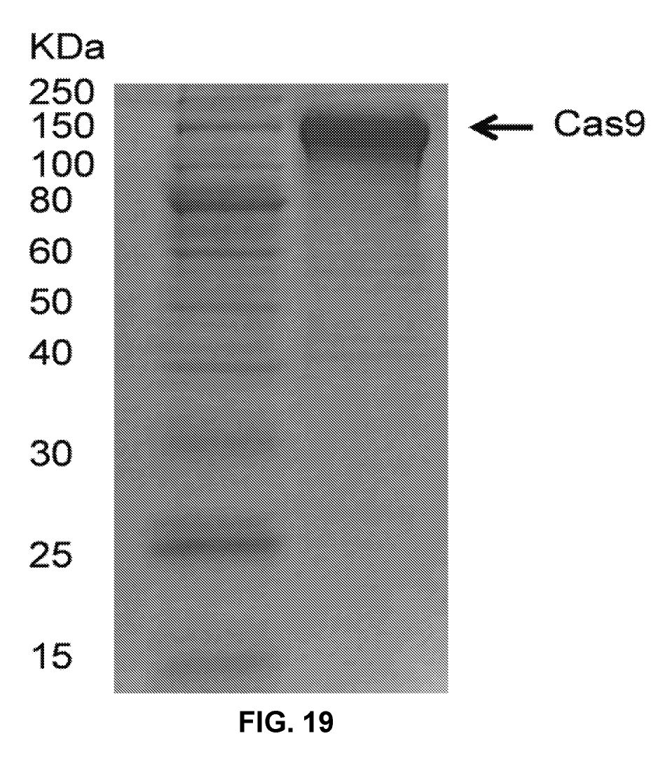

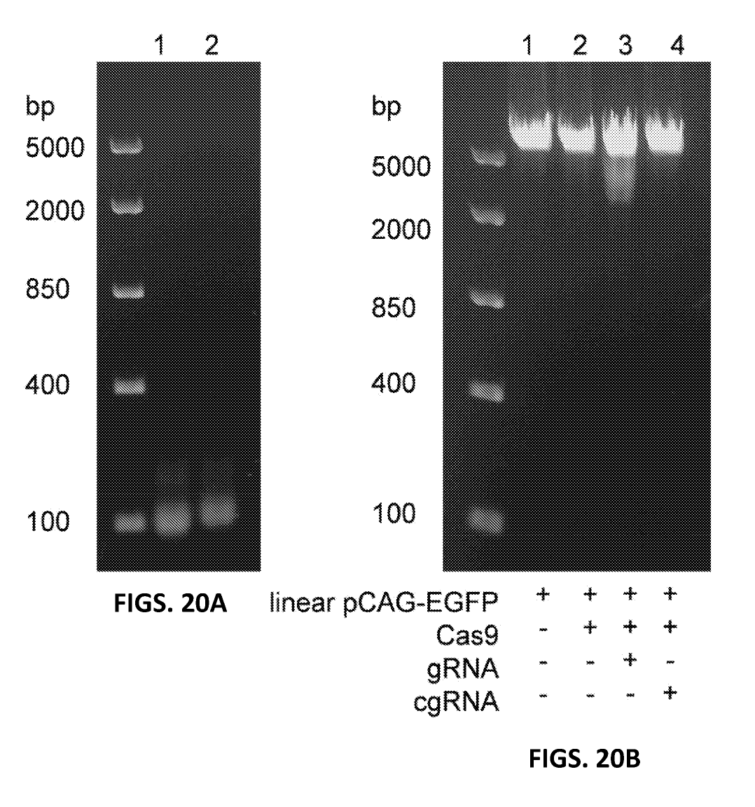

To demonstrate the DNA NC-mediated delivery of CRISPR-Cas9, the well-characterized and most extensively applied Streptococcus pyogenes Cas9 was selected.^{[17]} Recombinant Cas9 fused with N-terminal and C-terminal nuclear localization signals^{[18]} was purified following overexpression in Escherichia coli (FIG. 19) and incubated with one of two sgRNAs: one designed to target a sequence within the enhanced green fluorescent protein (EGFP) gene flanked by an NGG PAM, and the other control sgRNA (cgRNA) designed not to appreciably target any DNA sequence in EGFP or the human genome (FIG. 20A). It was confirmed that the resulting Cas9/sgRNA complex was active in vitro based on cleavage of a linearized plasmid encoding the EGFP gene, but only in the presence of Cas9 and the EGFP-targeting sgRNA (FIG. 20B).

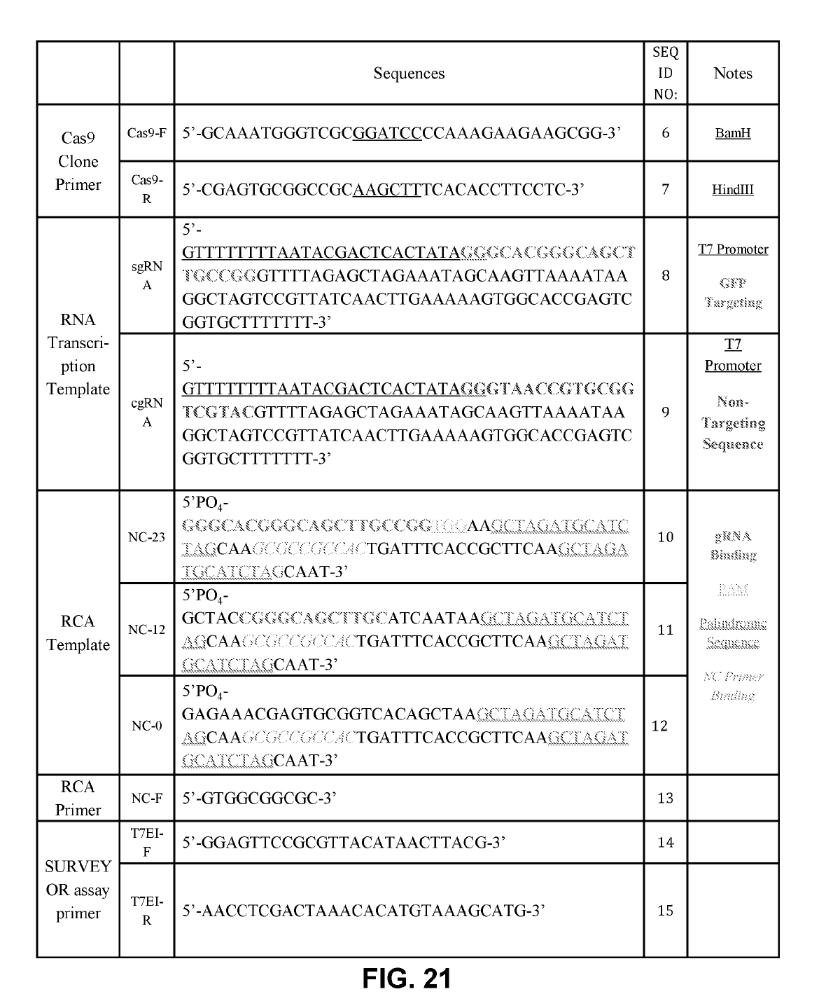

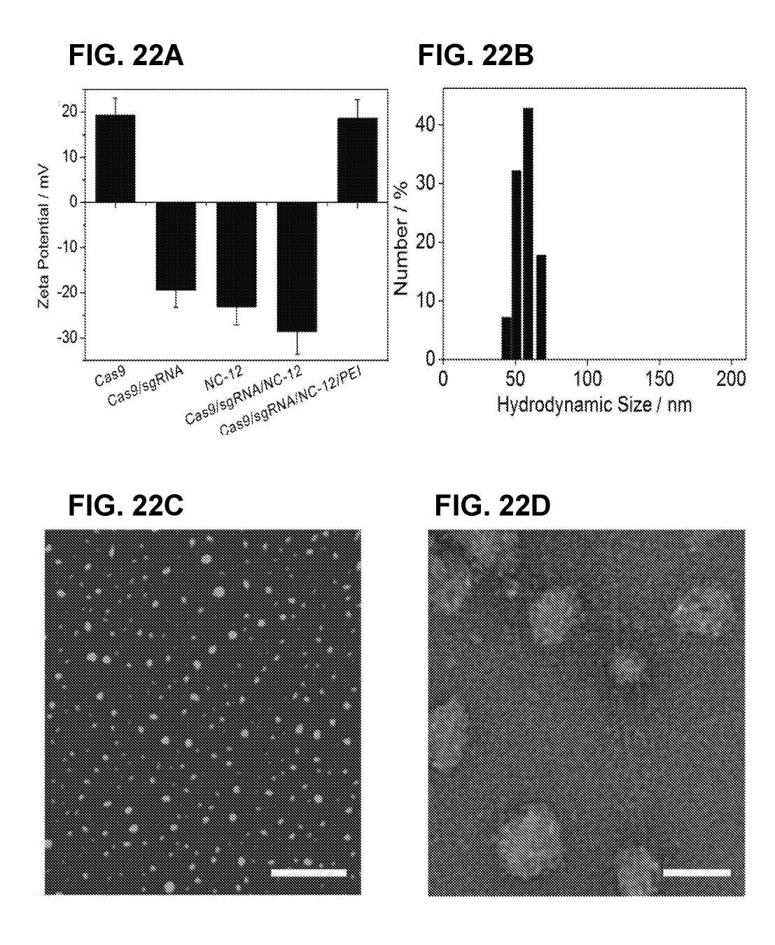

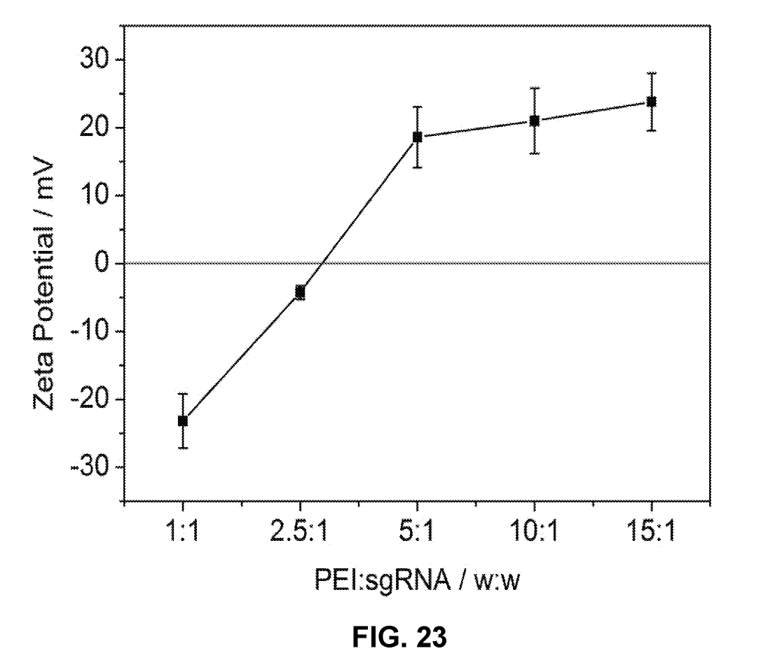

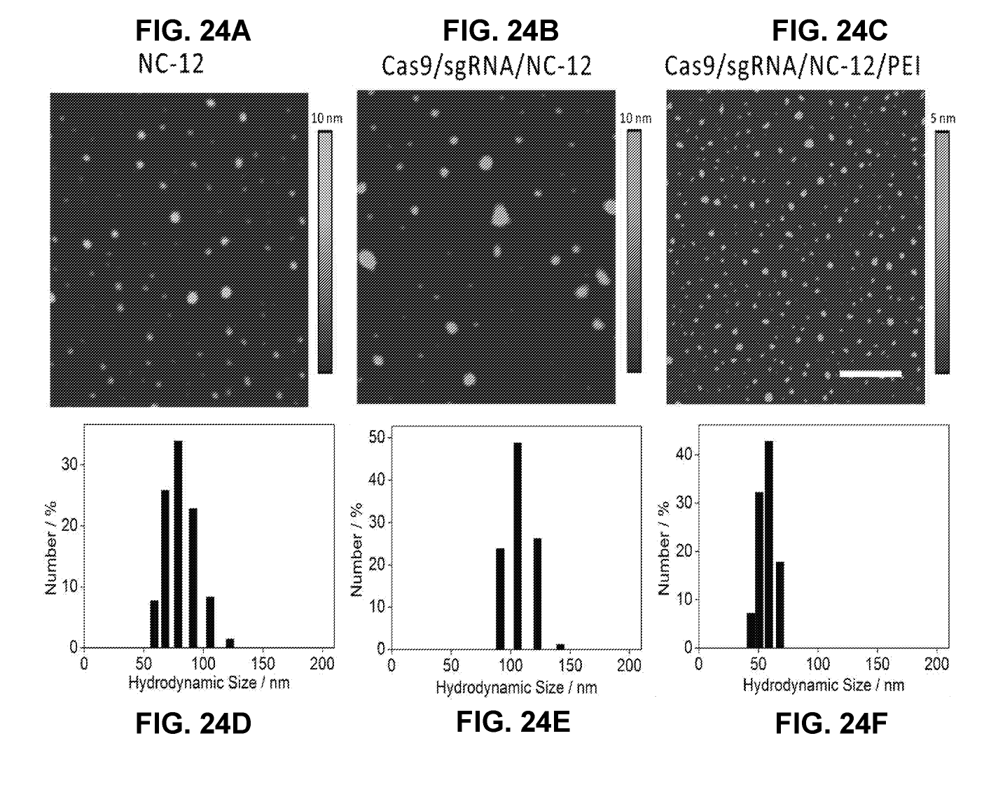

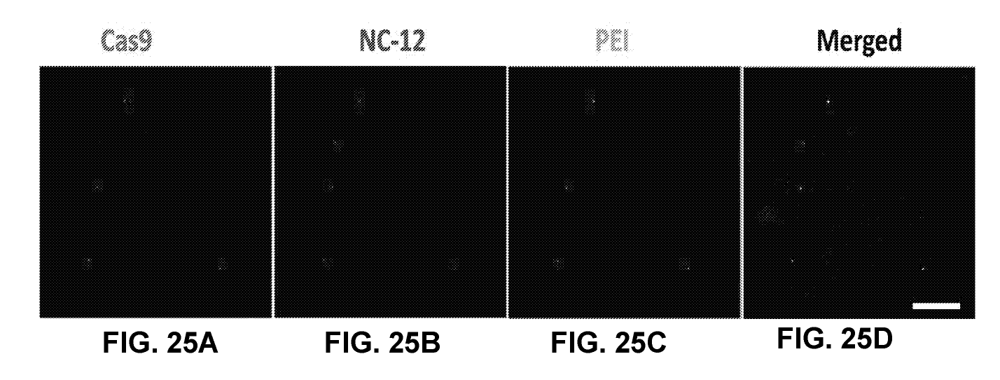

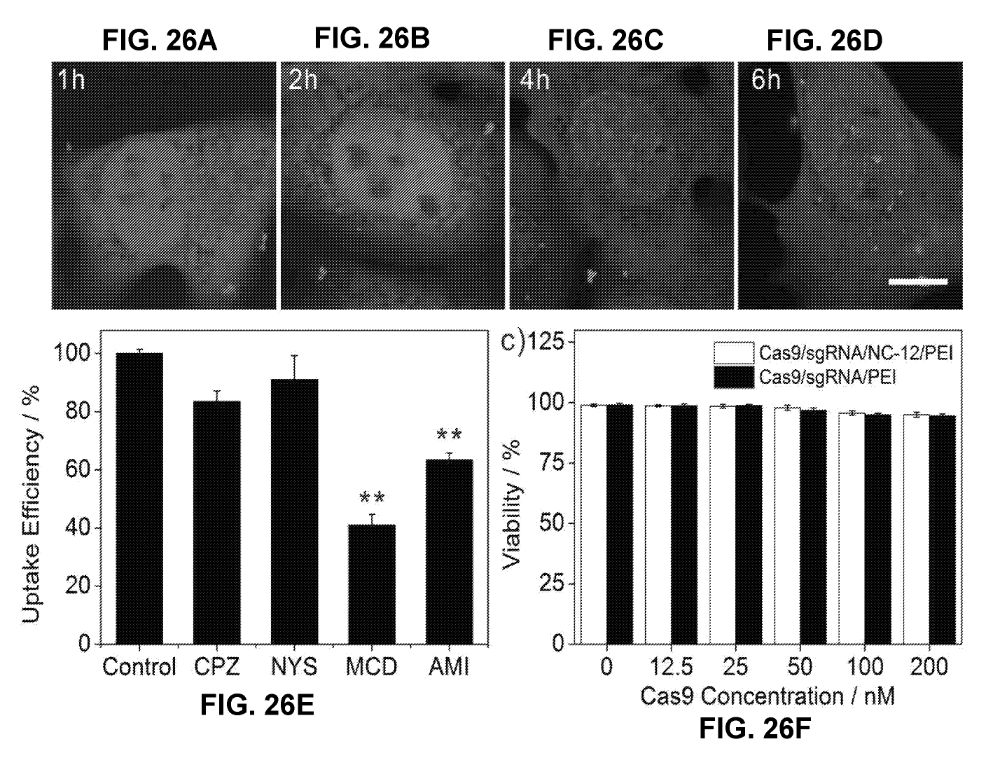

Next, the DNA NC to bind the Cas9/sgRNA complex was generated. The DNA template for RCA was designed to encode 12 nucleotides complementary to the 5′ end of the sgRNA (NC-12) along with the palindromic repeat that drives self-assembly (FIG. 21). The rationale was that the complementary sequence would promote base pairing between the DNA NC and the Cas9/sgRNA complex, thereby forming a strong but reversible interaction. To form the nanoparticle consisting of Cas9, sgRNA, NC-12, and PEI (Cas9/sgRNA/NC-12/PEI), Cas9 and the sgRNA were incubated together, followed by the addition of the NC-12, and then the addition of PEI. Measuring the zeta potential at each assembly step showed that the positively charged Cas9 (+19.3±3.8 mV) became negatively charged with the addition of sgRNA (−19.4±3.7 mV) and then NC-12 (−28.6±5 mV), which was reverted to positive charge upon the addition of PEI (+18.6±4.1 mV) (FIGS. 22A-22D and 23). Dynamic light scattering analysis (FIGS. 2B and 24A-24F), atomic force microscopy (FIG. 22C and FIGS. 24A-24F) and transmission electron microscopy (FIG. 22D) revealed that the Cas9/sgRNA/NC-12/PEI nanoparticles were uniformly sized with a hydrodynamic size of ˜56 nm. The fully assembled particle was observed to be more compact and uniformly sized than the NC-12 nanoclew and the Cas9/sgRNA/NC-12 complex, potentially due to offsetting the dispersing charges. To assess the co-localization of each component, we applied confocal laser scanning microscopy (CLSM) to image nanoparticles comprised of Cas9 labeled with Alexa Fluor 647 (AF647), the sgRNA, the NC-12 stained with Hoechst 33342, and PEI conjugated with FITC. Imaging revealed consistent co-localization of all dyes (FIGS. 25A-26D), confirming the stable assembly of Cas9/sgRNA/NC-12/PEI.

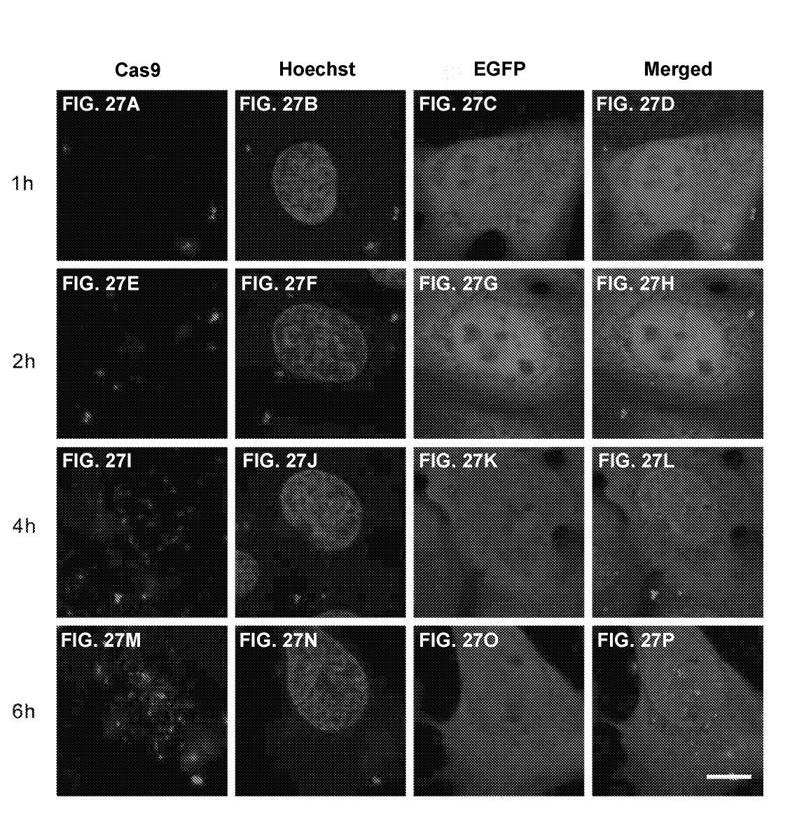

The ability of the particles to deliver Cas9/sgRNA into cultured cells was investigated. As a model, an established U2OS cell line that constitutively expresses a destabilized form of EGFP (U2OS.EGFP) was used.^{[6b]} CLSM, a technique with depth selectivity for analyzing subcellular location of delivered drugs,^{[14, 19]} was first applied to evaluate the localization of the Cas9/sgRNA/NC-12/PEI nanoparticles containing the AF647-labeled Cas9 (FIGS. 26A and 27A-27P). Over the course of six hours, the labeled Cas9 was observed to first bind to the cell surface, then enter the cytosol, and finally localize to the nuclei as indicated by the colocalization of the red fluorescence signal from AF647-Cas9 with the blue fluorescent signal of stained nuclei. To elucidate the mechanism of internalization, inhibitors of different endocytosis pathways^{[19b]} were applied and the relative uptake of the Cas9/sgRNA/NC-12/PEI nanoparticles containing AF647-labeled Cas9 was measured. Flow cytometry analysis revealed that the inhibitors methyl-β-cyclodextrin (MCD) and amiloride (AMI) imparted the greatest reduction in Cas9 uptake (FIG. 26E), suggesting that the particles were mainly internalized through lipid rafts and macropinocytosis.^{[19b]} Furthermore, the impact of the nanoparticles on cell viability was evaluated. TO-PRO-3 live/dead assay^{[7a]} demonstrated no measurable impact on viability even at high concentrations (200 nM) of Cas9 (FIG. 26F).

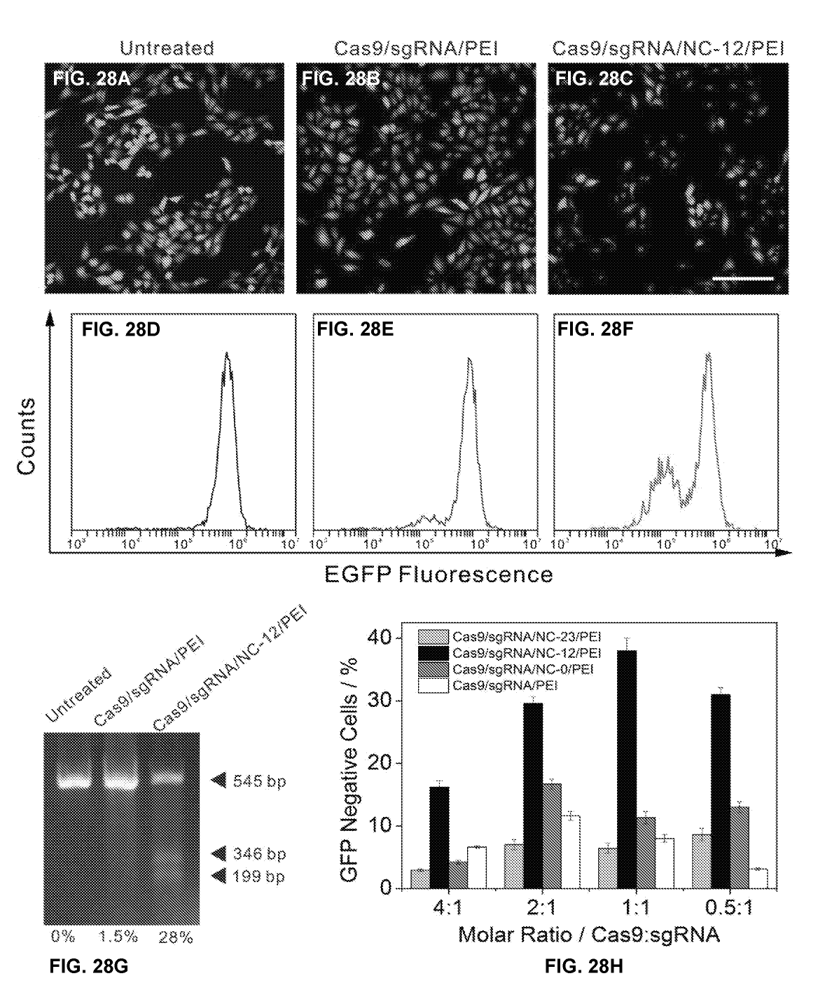

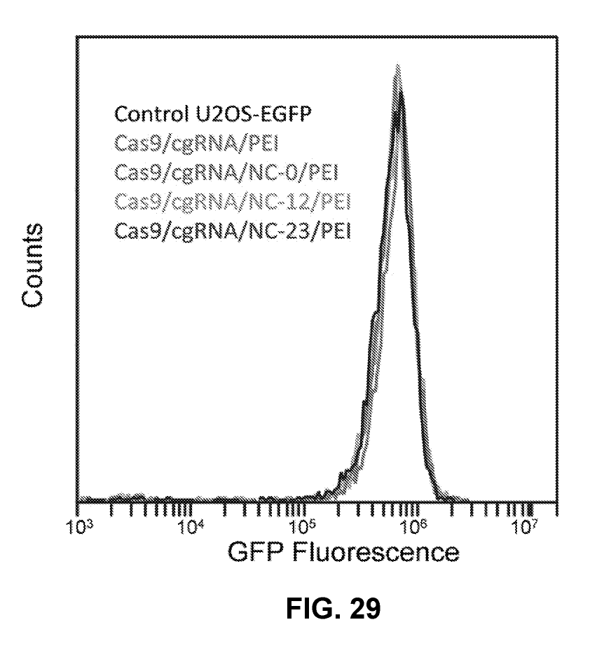

Based on the evidence that the Cas9/sgRNA would reach cell nucleus, the extent to which Cas9/sgRNA could drive the formation of indels through targeted DNA cleavage and repair by the endogenous NHEJ pathway was evaluated. By targeting the coding region of EGFP, most indels would shift the reading frame, thereby preventing proper EGFP expression. To evaluate the impact on EGFP expression, we incubated cells with the particles containing the EGFP-targeting sgRNA (Cas9/sgRNA/NC-12/PEI, FIGS. 28A-28F) or the non-targeting cgRNA (Cas9/cgRNA/NC-12/PEI, FIG. 29).

Fluorescence microscopy and flow cytometry analysis revealed that the sgRNA reduced fluorescence in ˜36% of the cells, whereas the cgRNA had a negligible effect in comparison to untreated cells. Particles prepared with only Cas9, sgRNA, and PEI were also evaluated and these particles were observed to reduce fluorescence in only 5% of the cells, demonstrating the importance of the DNA NC for effective delivery. To assess whether the reduction in fluorescence was attributed to indel formation, we applied the SURVEYOR assay that quantifies the frequency of mutations within an amplified target region.^{[3]} The assay revealed mutation frequencies of 28% and 1.5% for cells treated with Cas9/sgRNA/NC-12/PEI and Cas9/sgRNA/PEI (FIG. 28G), respectively, closely paralleling the flow cytometry analysis. We also subcloned the amplified target region of cells incubated with the Cas9/sgRNA/NC-12/PEI nanoparticles. Sanger sequencing of 20 clones revealed 7 clones with typical indels within the PAM or the sequence complementary to the sgRNA guide (FIG. 29), confirming the genetic disruption of EGFP expression by CRISPR-Cas9.^{[3]} One-time treatment with the DNA NC mediated Cas9/sgRNA delivery system lead to higher editing efficacy than the cell-penetrating peptides (CPPs) based vector (9.7%) if the variation of cell line and targeted locus were not taken into account.^{[7b]} Although the cationic lipid/anionic EGFP based delivery strategy showed higher editing efficacy (80%),^{[7a]} lipid-vehicles are often hampered by serum instability, which could be alleviated by polymer-based carriers.^{[8,20]}

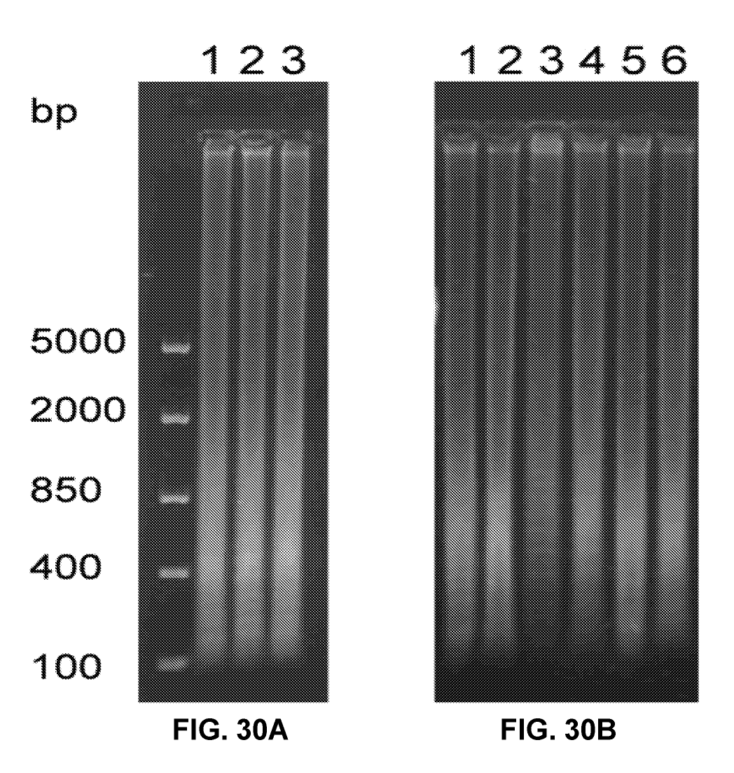

The impact the complementarity of the DNA NC and the sgRNA on the efficacy of Cas9-driven genome editing was next investigated. To address this, two additional variants of the DNA NC with 0 or 23 nucleotides complementary to the sgRNA (designated as NC-0 and NC-23, respectively) were generated. Agarose gel electrophoresis confirmed that NC-0 and NC-23 yielded similar molecular weight distributions as NC-12 and were resistant to Cas9/sgRNA degradation (FIGS. 30A and 30B). Subjecting the resulting particles to the U2OS.EGFP cells revealed that NC-12 yielded the highest fraction of EGFP negative cells (FIG. 28H). This trend was upheld for different molar ratios of Cas9 and the sgRNA, where the 1:1 standard stoichiometry of the Cas9/sgRNA complex yielded the greatest activity. Altogether, these results suggest that partial complementarity between the sgRNA and the NC may be important for efficient delivery, which may be attributed to the need for balancing Cas9/sgRNA loading and release.

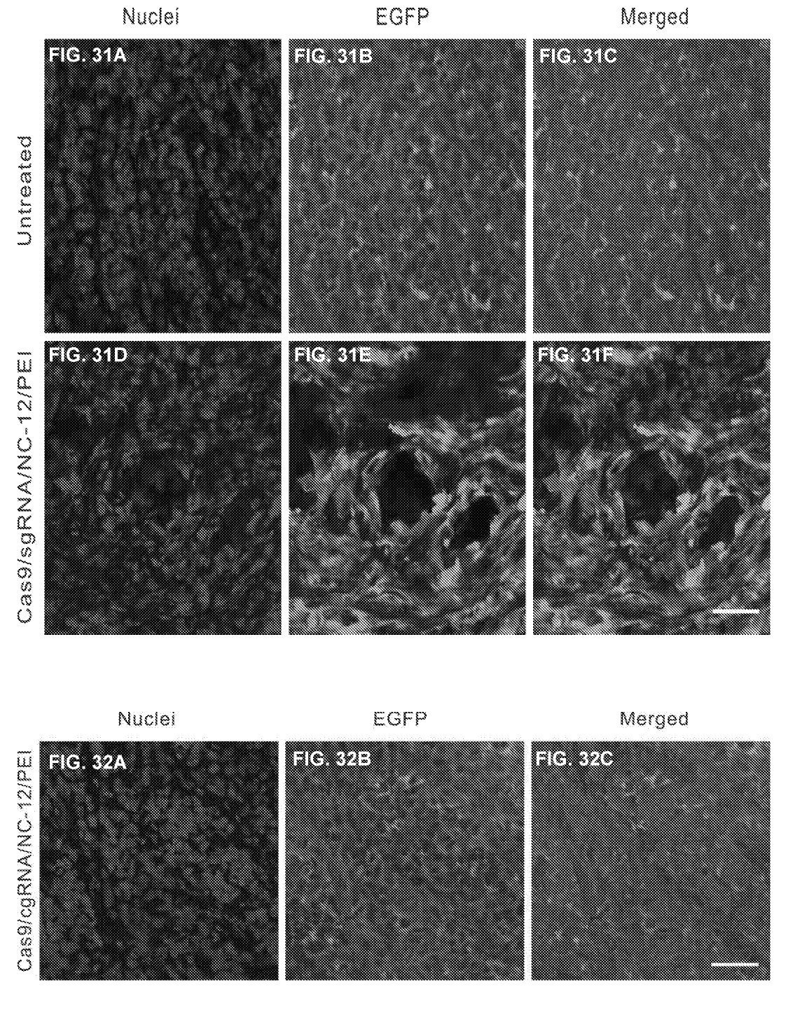

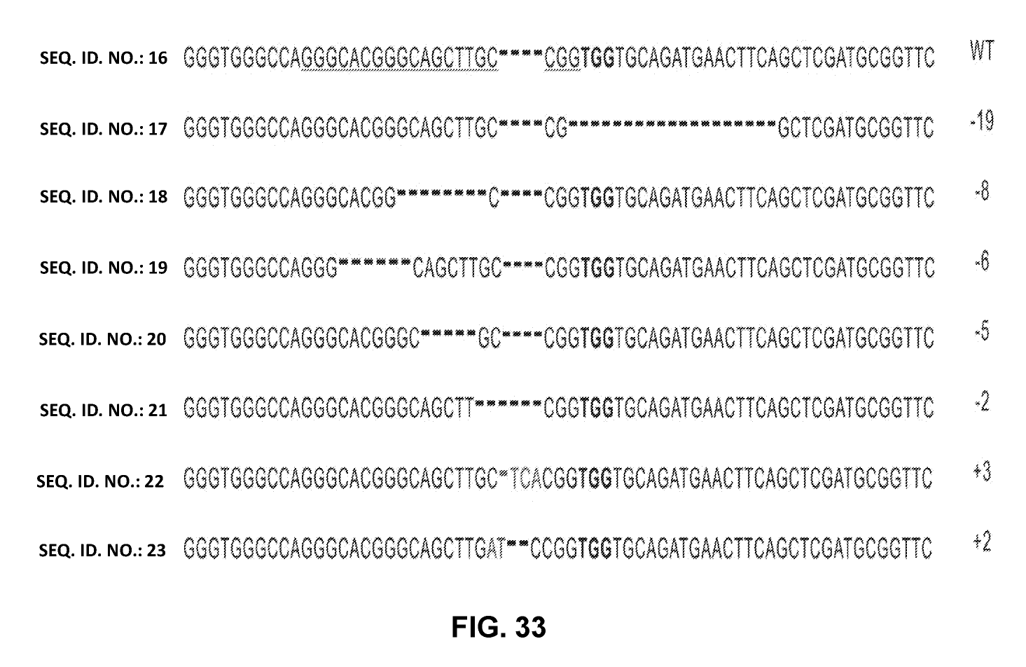

The in vivo EGFP disruption potency of Cas9/sgRNA delivered by NC-12 using U2OS.EGFP tumor bearing mice as models was next evaluated. 10 days after intratumoral injection, ˜25% the U2OS.EGFP cells in the frozen tumor sections near the site of injection lost EGFP expression in the Cas9/sgRNA/NC-12/PEI treated mice, while the tumors in the untreated group or the group treated with Cas9/cgRNA/NC-12/PEI did not show any loss of EGFP signal (FIGS. 31A-31F and 32A-32C).

In summary, a nanocage delivery vehicle to achieve targeted genome editing with CRISPR-Cas9 has been demonstrated in this Example. Our DNA NC-based delivery system represents, to our knowledge, the first example of a polymeric nanoparticle for the delivery of CRISPR-Cas9. The DNA NC pre-organized the Cas9/sgRNA into nanoparticles and increased the charge densities of the core in the core-shell assembly, which may have acted to stabilize the nanoparticle.^{[7a, 21]} Partial complementarity between the DNA nanoclew and the sgRNA guide sequence promoted the greatest extent of gene editing, potentially due to balancing binding and release of the Cas9/sgRNA complex by the nanoclew. Future implementation of the delivery vehicles may focus on attaching cell-specific targeting ligands,^{[22]} engineering the environmentally responsive release of the CRISPR-Cas9,^{[14, 23]} modifying the sequence of DNA NC to incorporate multiple sgRNAs for multiplexed editing, or employing the DNA NC or packaged DNA sequences as templates for homology-directed repair. The same NC architecture could also be used to incorporate other functional DNA-binding proteins, such as transcription factors, zinc-finger nucleases, and TALE nucleases, as well as other functional or protein-coding RNAs. The potential immunogenicity associated with DNA NCs should be further investigated for clinical translation.^{[24]}

Materials and Methods:

Materials.

All Chemicals were purchased from Sigma-Aldrich (St. Louis, Mo., USA) unless otherwise specified and were used as received. DNA oligonucleotides were purchased from Integrated DNA Technologies Inc. (Coralville, Iowa, USA). AmpliScribe™ T7-Flash™ Transcription Kit and CircLigase II ssDNA Ligase was purchased from Epicenter (Madison, Wis., USA). Bst 2.0 DNA polymerase and Pvul were purchased from New England BioLabs Inc. (Ipswich, Mass., USA). Plasmids pCAG-T3-hCAS-pA encoding the Cas9 protein (Addgene No. 48625) and pCAG-GFP encoding EGFP (Addgene No. 11150) were purchased from Addgene (Cambridge, Mass., USA). Linear polyethyleneimine (PEI) “max” (M.W. 40,000) was purchased from Polysciences Inc. (Warrington, Pa., USA).

Cloning, Expression and Purification of Cas9 Protein.

The human codon optimized S. pyogenes Cas9 gene with two nuclear localization sequences (NLS) at the N- and C-termini (Addgene 48625)^{[18]} was amplified and sub-cloned into pET-28a vector (Novagen) with primers Cas9-F/Cas9-R (FIG. 21), adding a N-terminal His₆-tag to the expressed Cas9. E. coli Rosetta (DE3) pLysS cells was transformed with pET28a-Cas9 and cultured for Cas9 expression. Briefly, a fresh E. coli colony was inoculated into 5 mL LB medium (supplemented with 10 μg/mL kanamycin and 34 μg/mL chloromycetin) and cultured at 37° C. overnight. The cell culture was then diluted with fresh LB medium by 100-fold and continued to culture for another 2-3 h until the OD₆₀₀ reached 0.6-0.8. 0.5 mM isopropyl β-D-1-thiogalactopy-ranoside (IPTG) was added to induce Cas9 expression at 20° C. for 8 h. The cells were then collected by centrifugation (4000×g, 15 min), suspended in Buffer A (20 mM Tris-HCl pH 8.0, 0.5 M NaCl, 10 mM imidazole and 1 mM PMSF) and lysed by sonication. Cell debris was removed by centrifugation (20000×g, 20 min) and the clear lysate was added to a column containing 1 mL Ni-NTA resin (Qiagen). After washing the column with Buffer B (20 mM Tris-HCl pH 8.0, 0.5 M NaCl and 60 mM imidazole), Cas9 was eluted with Buffer C (20 mM Tris-HCl pH 8.0, 0.5 M NaCl and 500 mM imidazole) and dialyzed against Buffer D (20 mM HEPES pH 7.4, 150 mM KCl, 1 mM DTT and 10% glycerol) at 4° C. overnight. The purified Cas9 was quantified by Bradford assay (Bio-Rad) and analyzed by SDS-PAGE.

Transcription and Purification of Single-Guide RNA (gRNA).

The sgRNA was transcribed in vitro with AmpliScribe™ T7-Flash® Transcription Kit (Epicentre) according to manufacturer's instructions. Transcription templates encoding a T7 promoter followed by the sgRNA were synthesized by IDT with the sgRNA containing a 20 bp EGFP targeting sequence and a control guiding RNA (cgRNA) that does not target EGFP or any genes in human genome (FIG. 21). The transcribed RNA was extracted by phenol:chloroform:IAA (Ambion) with Phase Lock Gel (5 Prime) to separate the RNA containing water phase. After removing unincorporated nucleotide with illustra Microspin G-50 columns (GE Healthcare), the RNA was ethanol precipitated and re-suspended in DEPC treated water. Purified RNA was analyzed by agarose gel electrophoresis and quantified with Nanodrop 2000c (Thermo Scientific).

Plasmid DNA Cleavage Assay to Detect Cas9 Activity.

Plasmid pCAG-GFP containing the EGFP gene (Addgene 11150)^{[25]} was linearized with Pvul (NEB), purified by GeneJET Gel Extraction Kit (Thermal Scientific) and used as the substrate for Cas9 activity assay. In a reaction volume of 20 μl containing NEBuffer 3 and linearized plasmid (300 ng), purified Cas9 (50 nM) and sgRNA (50 nM) were added. After digestion for 1 h at 37° C., the DNA was analyzed by 0.8% agarose gel electrophoresis.

Preparation and Characterization of DNA NC.

The DNA NCs were prepared by RCA using cyclized single stranded DNA (ssDNA) templates. 5′ phosphorylated linear ssDNA templates (FIG. 21) were cyclized by CircLigase II ssDNA ligase (Epicentre) following manufacture's instructions. Unligated ssDNA chains were removed with 1 U Exo I (NEB) at 37° C. for 45 min followed by heat inactivation at 80° C. for 15 min. The cyclized ssDNA template (10 pmol) was added into 1 mL 1× isothermal amplification buffer (NEB) together with 0.5 μM primer and 200 μM dNTP and heated to 95° C. for 5 min. After hybridizing the template and primer by cooling the mixture to room temperature, Bst 2.0 DNA polymerase (0.2 U/μL) was added to initiate the RCA. The RCA was performed at 65° C. overnight and the denatured polymerase after the reaction was removed by centrifugation at 14,000×g for 2 min. The supernatant was recovered and dialyzed against DI water using a Slide-A-Lyzer (20K MWCO, Thermo Scientific) for 48 h. The synthesized DNA NCs were analyzed by 0.8% agarose gel electrophoresis and Nanodrop 2000C (Thermal Scientific) was applied to measure the concentration and purity of the DNA NC. NC with high purity (A₂₆₀/A₂₈₀>1.8) was used for further studies. To evaluate the stability of NC, 300 ng NC was incubated with Cas9 (50 nM) and gRNA (50 nM) in NEBuffer 3 at 37° C. for 24 h and then analyzed using 0.8% agarose gel electrophoresis. Zeta potential and particle size of NC were measured with a Zetasizer (Malvern). To image the NC by atomic force microscopy (AFM), the NC was dropped and dried onto a silicon wafer (Ted Pella) and analyzed on a Nanoscope AFM instrument (Veeco, Santa Barbara, Calif.) using tapping mode in ambient air.

Assembly and Characterization of Cas9/sgRNA/NC/PEI.

Purified Cas9 and sgRNA at various molar ratios (4:1-0.5:1) were mixed in PBS and incubated at room temperature for 5 min, followed by the addition of DNA NC (NC:sgRNA weight ratio of 4:1) and incubated at room temperature for another 5 min. Afterwards, PEI “max” (Polysciences) was coated onto Cas9/sgRNA/NC at PEI:sgRNA weight ratio of 5:1 and equilibrated at room temperature for 5 min. The assemblies were further diluted to the concentration of sgRNA at 100 nM in deionized water for particle characterization or Opti-MEM medium (Life Technologies) for cell study. Size and zeta potential of Cas9/gRNA/NC/PEI were analyzed by a Zetasizer (Nano ZS, Malvern). AFM imaging was performed using a Nanoscope (Veeco, Santa Barbara, Calif.) on silicon wafer (Ted Pella) as described above. For TEM imaging, the Cas9/sgRNA/NC/PEI was dropped onto a TEM copper grid (300 mesh, Ted Pella) and stained with 2% uranyl acetate (w/v, in 50% ethanol). TEM images were observed on a JEM-2000FX (Hitachi) at 200 kV. The assembly was also visualized with confocal laser scanning microscope (CLSM, LSM 710, Zeiss) to confirm the colocalization of the components. Cas9 was conjugated with Alexa Fluor 647 C2 maleimide (AF647), PEI was conjugated with FITC NHS ester (Life Technologies) and the NC was stained with Hoechst 33342, a nucleic acid dye that stains only DNA but not RNA.^{[26]}

Cell Culture and EGFP Gene Disruption Assay.

The reporter cell line U2OS.EGFP with a single copy of destabilized EGFP gene integrated into the genome was a generous gift from Dr. J Keith Joung at Massachusetts General Hospital.^{[6b]} The cells were cultured in a 37° C. incubator under 5% CO₂ and 90% humidity with full serum medium: Dulbecco's Modified Eagle's Medium supplemented with 10% (v/v) FBS, 2 mM GlutaMAX (Life Technologies), penicillin (100 U/mL) and streptomycin (100 μg/mL). U2OS.EGFP cells were seeded into 24-well plates (˜25,000 cells per well) one day before the transfection. When the cells reached 70% confluence, the medium was replaced with 0.5 mL Opti-MEM medium (Life Technologies) containing the Cas9/gRNA loaded nanoparticles (gRNA concentration at 100 nM). After incubation for 4 h, the Cas9 containing medium was replaced with fresh full serum medium. Two days after the delivery, the cells were analyzed using a fluorescent microscope (IX71, Olympus). For the flow cytometry analysis, the cells were washed with ice cold PBS twice and trypsinized with 0.05% trypsin (Mediatech) at 37° C. for 1-2 min. Afterwards, the cells were washed and resuspended in full serum medium and analyzed by a BD Accuri C6 flow cytometer (BD Biosciences).

SURVEYOR Assay to Detect Genomic Modifications.

Genomic DNA of U2OS.EGFP cells was harvested 2 d after the delivery using GeneJET Genomic DNA Purification Kit (Thermo Scientific) according to manufacturer's instructions. The gRNA targeted genomic locus was amplified with Phusion Hot Start II High Fidelity DNA Polymerase (NEB) using primers T7EI-F/T7EI-R (FIG. 21). Touchdown PCR program ((98° C. for 10 s; 66-56° C. with −1° C./cycle for 15 s, 72° C. for 60 s) for 10 cycles and (98° C. for 10 s, 56° C. for 15 s, 72° C. for 60 s) for 25 cycles) was used to reduce non-specific amplifications. The amplicons were then purified by gel extraction and 200 ng of the purified DNA was added to 20-μL cleavage reaction containing 1×NEBuffer 2. After heating to 95° C. for 5 min, the mixture was cooled to form heteroduplex DNA. Afterwards, 1 μL T7EI (10 U/μl, NEB) was added and incubated at 7° C. for 15 min. The digested DNA was analyzed using 2% agarose gel electrophoresis. Indel formation efficiencies were calculated using Image J.

DNA Sequencing to Detect Genomic Mutations.

Purified PCR amplicons of the T7EI assay were cloned into Zero Blunt TOPO DNA sequencing vectors (Life Technologies). The cloned plasmids were purified by GeneJET Plasmid Miniprep Kit (Thermo Scientific) and sequenced by Eton Bioscience Inc. (RTP, NC, USA) with T7 promoter primer.

Determination of Endocytosis Pathways.

Cas9 was fluorescently labeled with AF647 to track its uptake. U2OS.EGFP cells were seeded in 24-well plates (˜25000 cells/well) and cultured for 2 d. Then the cells were pre-incubated with several endocytosis inhibitors^{[19b]}, such as chlorpromazine (CPZ, 10 μM) for clathrin-mediated endocytosis, nystatin (NYS, 25 μg/mL) for caveolin-mediated endocytosis, methyl-β-cyclodextrin (MCD, 3 mM) for lipid raft and amiloride (AMI, 1 mM) for macropinocytosis, for 1 h at 37° C., respectively. Afterwards, the cells were incubated with AF647-Cas9/sgRNA/NC/PEI for another 2 h in the presence of the inhibitors. Cells were then washed, trypsinized and resuspended in full serum medium, intracellular AF647 fluorescence intensities were analyzed by flow cytometry.

Intracellular Distribution of Cas9.

U2OS.EGFP cells were seeded in confocal dishes (MatTek) at a density of 1×10⁵ per well and cultured for 24 h. To image the nuclear accumulation of Cas9, the cells were incubated with AF647-Cas9/sgRNA/NC/PEI for 1 h, 2 h, 4 h and 6 h. After washing with 4° C. PBS twice, the cells were stained with Hoechst 33342 (1 μg/mL) for 10 min. Washed with cold PBS twice again, the cells were observed with CLSM immediately.

In Vitro Cytotoxicity.

U2OS.EGFP cells delivered with Cas9 were analyzed for cell survival using flow cytometry with TO-PRO-3 live/dead cell stain (Life Technologies).^{[7a]} Briefly, the U2OS.EGFP cells were seeded in 24-well plates (˜25000 cells/well) and cultured for 24 h. Then the cells were exposed to Cas9/sgRNA/NC-12/PEI and Cas9/sgRNA/PEI at different Cas9 concentrations for 4 h. Afterwards, the cells were washed with PBS and stained with TO-PRO-3 live/dead cell stain (1 μM) for 15 min. Washed, trypsinized and resuspended, the cells were then analyzed by flow cytometry.

In Vivo EGFP Disruption.

All animal experiments were conducted according to the Guide for Care and Use of Laboratory Animals approved by the Institutional Animal Care and Use Committee (IACUC) of University of North Carolina at Chapel Hill and North Carolina State University. To set up the U2OS.EGFP tumor model, the female nude mice (6 weeks, J:NU, The Jackson Laboratory) were subcutaneously inoculated with 1×10⁷ U2OS.EGFP cells. One mouse was inoculated with one tumor and when the volume of the tumors reached 200-400 mm³, the mice were intratumorally injected with 50 μL of the ˜56 nm nanoparticles (Cas9/sgRNA/NC-12/PEI or Cas9/cgRNA/NC-12/PEI) in PBS (Cas9 concentration at 5 μM). At day 10, the mice were euthanized and the tumors were collected, washed by saline thrice and followed by fixation in the 10% neutral buffered formalin. Tumor tissues within 5 mm of distance from the point of injections were sectioned. Cas9-mediated EGFP disruptions were visualized by staining the tumor sections with FITC conjugated GFP antibody (Thermo Scientific) and the nuclei were counterstained with Hoechst 33342. The stained slides were observed with CLSM.

Statistics.

All results were presented as Mean±SD Statistical analysis was performed using two-tailed student's t-test. The difference between experimental groups and control groups were considered statistically significant when p<0.05 or highly significant when p<0.01.

### REFERENCES FOR EXAMPLE 2

[1] Patrick D. Hsu, et al., Cell 2014, 157, 1262-1278.
[2] M. Jinek, et al., Science 2012, 337, 816-821.
[3] a) L. Cong, et al., Science 2013, 339, 819-823; b) P. Mali, et al., Science 2013, 339, 823-826.
[4] a) F. A. Ran, et al., Nature 2015, 520, 186-191; b) Randall J. Platt, et al., Cell 2014, 159, 440-455.
[5] M. A. Kotterman, D. V. Schaffer, Nat. Rev. Genet. 2014, 15, 445-451.
[6] a) V. Pattanayak, et al., Nat. Biotechnol. 2013, 31, 839-843; b) Y. Fu, J. A. et al., Nat. Biotechnol. 2013, 31, 822-826.
[7] a) J. A. Zuris, et al., Nat. Biotechnol. 2015, 33, 73-80; b) S. Ramakrishna, et al., Genome Res. 2014.
[8] Z. Gu, A. Biswas, M. Zhao, Y. Tang, Chem. Soc. Rev. 2011, 40, 3638-3655.
[9] T. U. Gerngross, Nat. Biotechnol. 2004, 22, 1409-1414.
[10] M. X. Sliwkowski, I. Mellman, Science 2013, 341, 1192-1198.
[11] M. Wade, Y.-C. Li, G. M. Wahl, Nat. Rev. Cancer. 2013, 13, 83-96.
[12] H. Yin, et al., Nat. Rev. Genet. 2014, 15, 541-555.
[13] a) M. M. Ali, et al., Chem. Soc. Rev. 2014, 43, 3324-3341; b) R. Hu, et al., Angew. Chem. 2014, 126, 5931-5936; Angew. Chem. Int. Ed. 2014, 53, 5821-5826; c) J. B. Lee, et al., Nat. Mater. 2012, 11, 316-322; d) H. Y. Lee, et al., Adv. Mater. 2015, 27, 3513-3517; e) R. J. Macfarlane, et al., Proc. Natl. Acad. Sci. U.S.A. 2014, 111, 14995-15000; f) M. R. Jones, N. C. Seeman, C. A. Mirkin, Science 2015, 347.
[14] W. Sun, et al, J. Am. Chem. Soc. 2014, 136, 14722-14725.
[15] G. Gasiunas, et al., Proc. Natl. Acad. Sci. U.S.A. 2012, 109, E2579-E2586.
[16] A. K. Varkouhi, et al., J. Control. Release 2011, 151, 220-228.
[17] J. A. Doudna, E. Charpentier, Science 2014, 346.
[18] W. Fujii, K. Kawasaki, K. Sugiura, K. Naito, Nucleic Acids Res. 2013, 41, e187.
[19] a) S. Bolte, F. P. CordeliÈRes, J. Microsc. 2006, 224, 213-232; b) R. Mo, et al., Nat. Commun. 2014, 5, 3364.
[20] C. Y. M. Hsu, H. Uludag, Nat. Protocols 2012, 7, 935-945.
[21] C. A. Hong, et al., Angew. Chem. 2015, 127, 6844-6848; Angew. Chem. Int. Ed. 2015, 54, 6740-6744.
[22] a) D. Peer, et al., Nat. Nanotechnol. 2007, 2, 751-760; b) E. K.-H. Chow, D. Ho, Sci. Transl. Med. 2013, 5, 216rv214.
[23] B. Zetsche, S. E. Volz, F. Zhang, Nat. Biotechnol. 2015, 33, 139-142.
[24] a) J. Tian, et al., Nat Immunol 2007, 8, 487-496; b) W. Sun, Z. Gu, Biomater. Sci. 2015, 3, 1018-1024.
[25] T. Matsuda, C. L. Cepko, Proc. Natl. Acad. Sci. U.S.A. 2004, 101, 16-22.
[26] H. Crissman, Z. Darzynkiewicz, R. Tobey, J. Steinkamp, Science 1985, 228, 1321-1324.

1. A nanocage comprising:
a single stranded (ss) nucleic acid molecule comprising one or more palindromic units, wherein the ss nucleic acid molecule is configured to self-assemble into the nanocage.

2. The nanocage of claim 1, further comprising a cargo molecule, wherein the cargo molecule is coupled to, encapsulated by, or coupled to and encapsulated by the nanocage.

3. The nanocage of claim 1 further comprising:
a nanocapsule comprising:
a release molecule; and
a stimuli responsive shell, wherein the stimuli responsive shell encapsulates the release molecule, and wherein the nanocapsule is coupled to or encapsulated by the nanocage.

4. The nanocage of claim 3, wherein the stimuli is pH.

5. The nanocage of claim 1, wherein the nucleic acid nanocage further comprises a targeting moiety, wherein the targeting moiety is coupled to the nanocage.

6. The nanocage of claim 2, wherein the cargo molecule is selected from the group consisting of: a nucleic acid; an amino acid; a peptide; a polypeptide; an antibody; a ribonucleoprotein; an aptamer; a ribozyme; a guide sequence for a ribozyme that is capable of inhibiting translation or transcription of essential tumor proteins and genes; a hormone; an immunomodulator; an antipyretic; an anxiolytic; an antipsychotic; an analgesic; an antispasmodic; an anti-inflammatory; an anti-histamine; an anti-infective; a chemotherapeutic; and any permissible combination thereof.

7. The nanocage of claim 2, wherein the cargo molecule is doxorubicin.

8. The nanocage of claim 7, wherein the targeting moiety is folic acid or an analogue thereof.

9. The nanocage of claim 8, wherein the ss nucleic acid molecule further comprises a plurality of GC-pair sequences.

10. The nanocage of claim 2, wherein the cargo molecule is a Cas9:sgRNA riboonucleoprotein complex.

11. The nanocage of claim 10, wherein the ss nucleic acid molecule is at least partially complementary to the sgRNA of the Cas9:sgRNA riboonucleoprotein complex.

12. The nanocage of claim 5, wherein the targeting moiety comprises a linker molecule operatively coupled to a targeting molecule, wherein the linker molecule is coupled to the nanocage.

13. The nanocage of claim 5, wherein the targeting moiety consists of a targeting molecule and wherein the targeting moiety is coupled to the nanocage.

14. The nanocage of claim 1, further comprising a surface modifier disposed around the nanocage.

15. The nanocage of claim 14, wherein the surface modifier generates an anionic, cationic, or neutral surface charge in one or more surface areas on the nanocage.

16.-58. (canceled)

59. A method comprising:
administering to a subject a nanocage comprising:
a single stranded (ss) nucleic acid molecule comprising:
one or more palindromic units, wherein the ss nucleic acid molecule is configured to self-assemble into the nanocage;
a cargo molecule, wherein the cargo molecule is coupled to, encapsulated by, or coupled to and encapsulated by the nanocage; and
a nanocapsule comprising:
a release molecule; and
a stimuli responsive shell, wherein the stimuli responsive shell encapsulates the release molecule, and wherein the nanocapsule is coupled to or encapsulated by the nanocage.

60. The method of claim 59, wherein the cargo molecule is selected from the group consisting of: a nucleic acid; an amino acid; a peptide; a polypeptide; an antibody; a ribonucleoprotein; an aptamer; a ribozyme; a guide sequence for a ribozyme that is capable of inhibiting translation or transcription of essential tumor proteins and genes; a hormone; an immunomodulator; an antipyretic; an anxiolytic; an antipsychotic; an analgesic; an antispasmodic; an anti-inflammatory; an anti-histamine; an anti-infective; a chemotherapeutic; and any permissible combination thereof.

61. The method of claim 59, wherein the cargo molecule is a chemotherapeutic and the subject has a cancer.

62. The method of claim 59, wherein the cargo molecule is a Cas9:sgRNA ribonucleprotein complex and the ss nucleic acid molecule is at least partially complementary to the sgRNA of the Cas9:sgRNA ribonucleoprotein complex.

63. A method of genome editing comprising:
contacting a cell with an amount of a nanocage comprising:
a single stranded (ss) nucleic acid molecule comprising one or more palindromic units, wherein the ss nucleic acid molecule is configured to self-assemble into the nanocage;
a cargo molecule, wherein the cargo molecule is coupled to, encapsulated by, or coupled to and encapsulated by the nanocage, wherein the cargo molecule is a Cas9:sgRNA ribonucleprotein complex and the ss nucleic acid molecule is at least partially complementary to the sgRNA of the Cas9:sgRNA ribonucleoprotein complex; and
a nanocapsule comprising:
a release molecule; and
a stimuli responsive shell, wherein the stimuli responsive shell encapsulates the release molecule, and wherein the nanocapsule is coupled to or encapsulated by the nanocage.
